(19) United States
(12) Patent Application Publication
(10) Pub. No.: US 2021/0251555 A1
(43) Pub. Date: Aug. 19, 2021

(54) Stroke Rehabilitation Method and System Using a Brain-Computer Interface (BCI)

(71) Applicant: RehabSwift Pty Ltd, St Morris (AU)

(72) Inventor: Sam Darvishi, St Morris (AU)

(21) Appl. No.: 17/265,235

(22) PCT Filed: Aug. 3, 2018
(86) PCT No.: PCT/AU2018/000128

§ 371 Date: Feb. 2, 2021

(51) Int. Cl. A61B 5/375; A61B 5/378; A61B 5/38; A61B 5/16; A61B 5/31; A61H 1/02; A61B 5/00; G16H 20/70; G06N 3/00

(52) U.S. Cl. CPC A61B 5/375; A61B 5/378; A61B 5/38; A61B 5/162; A61B 5/31; A61N 2/006; A61B 5/742; G16H 20/70; G06N 3/004; A61B 2505/09; A61H 1/0288

(57) ABSTRACT

A Brain-Computer Interface (BCI) based rehabilitation system and method is described in which an auditory or visual stimulus is provided to a user instructing them to imagine performing a physical action with a body part such as a hand during a trial period. A BCI processes the electroencephalography (EEG) signals to perform feature extraction and then feature translation (classification) to determine if the user intended to perform the action. If the intension was detected the body part is incrementally moved to provide proprioceptive feedback to the user. The feedback process is repeated at a Feedback Update Interval (FUI) of 100 ms or less. Preferably a reaction time test is used to determine the optimal FUI for an individual where shorter FUIs used for shorter reaction times. In one embodiment, if the user has slow reaction times, the FUI is initially between 100 ms and 1000 ms and gradually decreased over a series of sessions until the FUI is less than 100 ms.

### CROSS-REFERENCE TO RELATED APPLICATION

This application is the United States national phase of International Application No. PCT/AU2018/000128 filed Aug. 3, 2018, the disclosure of which is hereby incorporated by reference in its entirety.

### BACKGROUND OF THE INVENTION

### Technical Field

The present disclosure relates to rehabilitation systems. In a particular form the present disclosure relates to stroke rehabilitation using a BCI.

### Description of Related Art

According to World Health Organization, 15 million people suffer stroke each year, where almost one third thereof do not adequately recover after stroke. One of the major stroke aftermaths is hemiparesis of the upper limbs and impairment of the arm motor functions. Rehabilitation of the hand motor functions following stroke is a key element to properly performing activities of daily living. However, traditional stroke rehabilitation techniques such as physiotherapy do not provide sufficient improvement to at least 30% of stroke victims

A range of alternative treatments have been proposed to address this gap. One approach is the use of a Brain-Computer Interface (BCI) that seeks to exploit motor imagery (MI). A BCI is a system that measures central nervous system (CNS) activity and converts it into artificial output that replaces, restores, enhances, supplements or improves natural CNS output, and thereby changes the ongoing interactions between CNS and its external or internal environment. The activity of CNS can be measured via sensors mounted on the scalp, on the brain's surface or implanted within the brain. The BCI measures the signals arising from CNS activity, extracts their related features, and then translates them into commands readable by computers/machines.

Motor imagery (MI), that is imagining performing an action, activates the brain in a similar manner to the actual movement. These similar phenomena are associated with a decrease in the spectral power of sensorimotor rhythms that occur within 8-30 Hz frequency band, which can be measured using electroencephalography (EEG) sensors. This decrement in spectral power of EEG signals recorded over the motor cortex is referred as event related desynchronization (ERD) and is followed by a spectral power rebound effect referred to as event related synchronization (ERS). These phenomena mainly occur within the hemisphere contralateral to the performed or imagined hand movement.

BCI systems that exploit MI offer a unique opportunity to activate the perilesional brain areas of the damaged hemisphere for those 30% of stroke patients who do not benefit from physiotherapy. This brain activation, if properly coupled with real time sensory feedback, closes the sensorimotor loop, and thus may promote Hebbian-like neuroplasticity following stroke. Therefore, Brain-Computer Interfaces (BCI) have been employed to enhance stroke rehabilitation through harnessing the power of neuroplasticity by i) monitoring MI occurrence through screening movement related potentials and, ii) providing sensory feedback to close the sensorimotor loop.

One difficulty is the occurrence of stroke in different cortical and subcortical areas of the brain may disrupt movement preparation and motor imagery and weaken the ERD to different extents. This makes application of a MI based BCI system more challenging as the signals are weaker or generation mechanisms may be damaged or impaired. Most BCI systems for stroke rehabilitation have been based on operant conditioning. In operant (instrumental) conditioning, implementing a specific behaviour is reinforced through provision of a reward. A myriad of techniques have been employed to capitalize on operant learning for promoting stroke recovery following stroke:

i) in the most common method a cursor position update on a monitor is used as visual feedback to reward down-regulation of sensorimotor rhythms;

ii) in another method that largely capitalizes on activation of the mirror neurons system (MNS), movement of a virtual arm is given to reward ERD modulation;

iii) proprioceptive feedback provision via an orthosis, a robot, or by Functional Electrical Stimulation (FES) has also been used to reward self-regulation of the brain oscillatory patterns for stroke rehabilitation.

However, operant learning of sensorimotor rhythms regulation is usually a long and time consuming procedure even with healthy populations. Thus, this length of training time may be an important barrier for administering neurofeedback training with stroke patients who may lose motivation if they do not gain the sense of autonomy in a timely manner. Moreover even when stroke patients learned ERD modulation through operant learning, no behavioural gain was reported.

Another approach has sought to exploit Hebbian learning, also known as neuroplasticity. In 1949, Donald Hebb postulated if input from neuron A, contributes to activation of neuron B, the synaptic connection from neuron A to neuron B will be strengthened. This form of synaptic plasticity that is based on the correlation between neurons' activity is referred as activity-dependent Hebbian plasticity. Using this learning rule, both enhancement and weakening of the synaptic connections between neurons can be explained. More specifically, if the activity of two neurons (or group of neurons) is correlated, then long term potentiation (LTP) occurs and manifests itself by strengthening the synaptic efficacy between co-activated neurons. However, if activity between two neurons (or ensemble of neurons) is dissociated, then long term depression (LTD) occurs that weakens the synaptic connection between dissociated neurons.

In the realm of BCI application for motor rehabilitation following stroke, the activity dependent plasticity has been studied by a number of laboratories. One group investigated the effect of this learning rule through accurately coupling MRCP elicited from imagination of a simple dorsiflexion movement with afferent feedback using peripheral electrical stimulation to the common peroneal nerve. They reported that significant plasticity was induced only when the afferent volley was timed to arrive during the peak negativity of the MRCP generated during the imagined task. In this paradigm, appropriately timed afferent input activates cortico-cortical projections from sensory cortex to the primary motor cortex (M1) at a time that M1 is active (through imagery), results in strengthening of the sensorimotor pathways. This may explain why they recorded stronger motor evoked potentials that reflect the corticospinal excitability, following the neurofeedback training sessions, compared to the control values. Another group have shown that learning sensorimotor desynchronization leads to behavioural gains after neurofeedback training using a brain-robot interface. They reported that the acquired skill for sustained Beta-ERD was correlated significantly with subsequent motor improvement. In another study of the same group, they reported that the robotic feedback to motor-imagery related sensorimotor Beta-ERD induced robust and muscle-specific changes of corticospinal excitability. Therefore, another promising treatment approach is to utilise activity-dependent Hebbian plasticity within therapeutic BCI paradigms.

However whilst primary (ie proof of principal) applications of BCI for stroke rehabilitation have provided promising results, the widespread application and dissemination of BCI for stroke rehabilitation necessitates its optimization to provide clinically significant outcomes in a timely and cost efficient fashion. There is thus a need to provide improved BCI based rehabilitation methods and systems, to provide patients with a useful alternative to existing methods and systems.

### SUMMARY OF THE INVENTION

According to a first aspect there is provided a Motor Imagery (MI) based Brain-Computer Interface (BCI) rehabilitation method, the method comprising:

performing a plurality of trials wherein each trial comprises:
providing an auditory or visual stimulus to a user to instruct them to imagine performing a physical action with a body part during a trial period;
periodically processing one or more BCI input signals from one or more sensors configured to record the electrical or magnetic activity of the brain or the brain metabolism during the trial period at a Feedback Update Interval (FUI), wherein the Feedback Update Interval is 100 ms or less, and processing the one or more signals is performed in a time less than the FUI and comprises:
   determining if a Motor Imagery (MI, intention to perform the instructed action) was formed during a sampling window; and
   generating a BCI output signal to actuate an output apparatus to move the user's body part if it is determined that a MI was formed to provide proprioceptive feedback to the user.

The above method thus detects MI and provides both proprioceptive feedback, via the actuated output apparatus, as well as visual feedback to the user, as the user can observe passive movement of the body part engaged with the output apparatus. The movements may be incremental so that over a series of sampling windows gross movement of the body part occurs for example a hand moving from a flexed to an extended position.

In one form, the method further comprises the steps of measuring a reaction time of the corresponding unaffected limb or part of a limb of the user prior to performing one or more trials, and determining the FUI interval for the one or more trials based upon the measured reaction time, wherein reaction times are positively correlated with FUI values such that shorter reaction times generate shorter FUIs. In a further form, determining the FUI for the one or more trials comprises classifying the reaction time into one of a plurality of reaction time ranges, and each reaction time range has an associated FUI value. In a further form, the plurality of trials are broken into a plurality of sessions, each session comprising a plurality of trial runs, and each trial run comprises a set of consecutive trials using the same FUI, and the method further comprises obtaining a measure of improvement after a session, and decreasing the FUI value for a subsequent session if the measure of improvement exceeds an improvement threshold until the FUI value reaches a lower limit, where the lower limit is determined from the measured reaction time.

In one form, the plurality of trials are broken into a plurality of sessions, each session comprising a plurality of trial runs, and each trial run comprises a set of consecutive trials using the same FUI, and the method further comprises the step of measuring the reaction time of the affected limb or part of affected limb of the user if there is residual motor function in the affected limb or part of affected limb, and if the measured reaction time is greater than a first threshold, then the FUI interval is set to an initial FUI value between 100 ms and an upper value, and if there is no residual motor function in the affected limb or part of affected limb, the FUI value is set to the upper value, and the method further comprises obtaining a measure of improvement after one or more sessions, and if the measure of improvement reaches a threshold value then the FUI is reduced until the FUI value is less than 100 ms. In a further form reaction times are measured between sessions, and if the reaction time decreases the FUI for the next session is decreased.

In one form the consecutive trials, now labelled motor imagery trials, within a run are interspersed with relaxation trials during which the user does not imagine moving the body part, and Event Related Desynchronisation (ERD) times are calculated based on the difference between the spectral power of the motor imagery trials and relaxation trials within a trial run and one of the measures of improvement is based on the ERD during the trial run.

In one form each relaxation trial comprises:

providing an auditory or visual stimulus to a user to instruct them to relax by performing a relaxation task, such as concentration on their breathing;

periodically processing one or more signals from one or more sensors configured to record the electrical or magnetic activity of the brain or the brain metabolism during the trial period at a Feedback Update Interval (FUI), wherein the Feedback Update Interval is 100 ms or less, and processing the one or more signals is performed in a time less than the FUI and comprises:
determining if the instructed relaxation was performed during a sampling window; and
generating a BCI output signal to move a non engaged visual feedback component of the output apparatus and/or to provide auditory feedback to the user if it is determined that the relaxation task was performed.

In one form the measurement of improvement is based on an accuracy measure based upon the number of trials where the user exceeds a threshold level of movement of the body part.

In one form obtaining the measure of improvement comprises taking a plurality of measurements of an active motor evoked potential (MEP) of the user.

In a further form the body part is a hand, and obtaining the measure of improvement comprises

obtaining a plurality of measurements of active MEP, wherein each measurement comprises:
providing an auditory stimulus to a user to instruct them to extend one or more fingers;
measuring the amount of finger extension force by the one or more fingers;
providing feedback to the user indicating the amount of finger extension force measured and a desired range;
triggering a transcranial magnetic stimulation machine to stimulate a target muscle in the user when the measured finger extension force falls within the desired range and triggering an electromyogram (EMG) amplifier to record an active MEP;
measuring the peak-to-trough value of the active MEP;

obtaining an estimate of the average active MEP using the plurality of the peak-to-trough values.

In a further form the desired range is between 10-10000 grams of force. In a further form the desired range is between 10-1000 grams of force.

In a further form, measuring a reaction time comprises measuring a reaction time using a simple reaction time test in which a stimulus is provided to the user, wherein the stimulus may be a visual, auditory, haptic, proprioceptive, or any combination thereof.

In a further form, measuring a reaction time comprises measuring a reaction time using a choice reaction time test in which plurality of stimuli are provided to the user, wherein the stimuli may be a visual, auditory, haptic, proprioceptive, or any combination thereof.

In a further form, the reaction time is calculated as the difference between the time of stimulus or stimuli exposure and the onset of observing a significant increase within a time window of up to 1000 ms after the time of stimulus or stimuli exposure in one or more of:

an amplitude of an electromyogram (EMG) signal of the target muscle,

an area under a curve of the absolute EMG signal of the target muscle; or

an area under a curve of the squared EMG signal of the target muscle.

In one form determining if the motor imagery of the instructed action was formed during a sampling window comprises detecting event related desynchronization (ERD) in the sensorimotor cortex using the one or more BCI input signals from one or more sensors.

In one form, the one or more BCI input signals from one or more sensors are electroencephalography (EEG) signals from a plurality of EEG sensor electrodes placed on the skull of the user.

In one form, determining if the Motor Imagery (intention to perform the instructed action) was formed during a sampling window comprises pre-processing the one or more signals to reduce noise and/or artefacts, performing feature extraction on the pre-processed one or more signals, post-processing extracted features to improve feature distribution and/or mitigate redundancy, and using a feature translator to determine if the extracted features indicate the Motor Imagery (intention to perform the instructed action) was formed during the sampling window.

In a further form pre-processing is performed using an 8-30 Hz band-pass filter and a spatial filter, feature extraction is performed using either an autoregressive (AR) model with at least 10 orders, or a continuous wavelet transform, or a fast Fourier transform, and the feature translator performs classification of the extracted features, using one or more of a linear regression model, a linear discriminant analysis, a support vector machine, or an adaptive neuro-fuzzy inference system (ANFIS) classifier to classify whether an event related desynchronization (ERD) in the 16-30 Hz (β) band occurred during the sample window.

In one form, the sampling window is between 100ms and 2000 ms inclusive and ends just prior to the start of processing the one or more signals. In a further form the sampling window is between 750 ms and 1000 ms (inclusive).

In one form, the BCI output signal is a binary signal, and the output apparatus incrementally moves the body part if a first signal is received, and the output apparatus does not moves the body part if a second signal is received. In a further form, the body part is a hand, and the output apparatus is a servomotor controlled flexible orthosis configured to support a hand and move fingers from a fully flexed position to a fully extended position in a series of incremental steps.

According to a second aspect, there is provided a Motor Imagery (MI) based Brain-Computer Interface (BCI) rehabilitation system comprising:

one or more sensors configured to record the electrical or magnetic activity of the brain or the brain metabolism during the trial period and generate one or more BCI input signals;

a computing apparatus comprising an output indicator device, a processor and a memory; and

an output apparatus in communication with the computing apparatus and comprising a body part support and a motor configured to incrementally move the body part support between two positions in response to one or more BCI output signals received from the computing apparatus,

wherein the BCI input signals are provided as input to the computing apparatus, and the memory comprises instructions to configure the processor to perform a plurality of BCI trials according to the method of the first aspect.

In one form the one or more sensors configured to record the electrical or magnetic activity of the brain or the brain metabolism during the trial period comprises:

a wearable apparatus comprising a plurality of electroencephalography (EEG) sensor electrodes;

an amplifier configured to receive and amplify the signals from the plurality of EEG sensor electrodes to generate the one or more BCI input signals. The amplifier may also perform signal conditioning, sample and hold, and signal processing of the EEG sensor signals.

In one form the body part is a hand, and the motor is a servomotor and the body part support is a servo motor controlled flexible orthosis configured to support a hand and move fingers from a fully flexed position to a fully extended position in a series of incremental steps.

In one form the output apparatus further comprises a visual feedback component comprising a servomotor controlled flexible orthosis configured to move a flexible member, without being engaged with any body part, from a fully flexed position to a fully extended position in a series of incremental steps.

In one form the system further comprises a force sensor, a transcranial magnetic stimulation machine, and an electromyogram amplifier.

According to a second aspect, there is provided a computer readable medium comprising instructions for causing a processor to perform the method of the first aspect.

### BRIEF DESCRIPTION OF DRAWINGS

Embodiments of the present disclosure will be discussed with reference to the accompanying drawings wherein:

FIG. 1A is a flowchart of a Motor Imagery (MI) BCI based rehabilitation method according to an embodiment;

FIG. 1B is a schematic diagram of a MI BCI based rehabilitation system according to an embodiment;

FIG. 2 is a schematic diagram of trial sequence of a MI BCI based rehabilitation method according to an embodiment;

FIG. 3A is a schematic plot showing the change in Feedback Update Interval (FUI) over a series of trials according to an embodiment;

FIG. 3B is a schematic plot showing the correlation between Feedback Update Interval (FUI) and reaction time according to an embodiment;

FIG. 4A is a scatter plot of reaction time and BCI accuracy with a FUI of 16 ms according to an embodiment;

FIG. 4B is a scatter plot of reaction time and BCI accuracy with a FUI of 96 ms according to an embodiment;

FIG. 4C is a two way ANOVA plot for BCI accuracy for a group of poor imagers and a group of good imager at FUIs of 16 ms and 96 ms according to an embodiment;

FIG. 4D is a two way ANOVA plot of BCI Information Transfer Rates for a group of poor imagers and a group of good imager at FUIs of 16 ms and 96 ms according to an embodiment;

FIG. 4E is a plot of poor imagers' Event Related Desynchronization (ERD) percentages across different ECG frequency bands for FUIs of 16 ms and 96 ms according to an embodiment;

FIG. 4F is a plot of good imagers' Event Related Desynchronization (ERD) percentages across different ECG frequency bands for FUIs of 16 ms and 96 ms according to an embodiment;

FIG. 5A is a schematic plot of a trial of a BCI rehabilitation method according to an embodiment;

FIG. 5B is a plot of Action Research Arm Test (ARAT) scores for a stroke patient over the course of the trial illustrated in FIG. 5A according to an embodiment;

FIG. 5C is a plot of Maximum Voluntary Contraction (MVC) calculated using root mean squares of EMG signals for a stroke patient over the course of the trial illustrated in FIG. 5A according to an embodiment;

FIG. 5D is a plot of Resting Motor Evoked Potential (MEP) scores for a stroke patient over the course of the trial illustrated in FIG. 5A according to an embodiment; and

FIG. 5E is a plot of Active Motor Evoked Potential (MEP) scores for a stroke patient over the course of the trial illustrated in FIG. 5A according to an embodiment.

In the following description, like reference characters designate like or corresponding parts throughout the figures.

### DESCRIPTION OF THE INVENTION

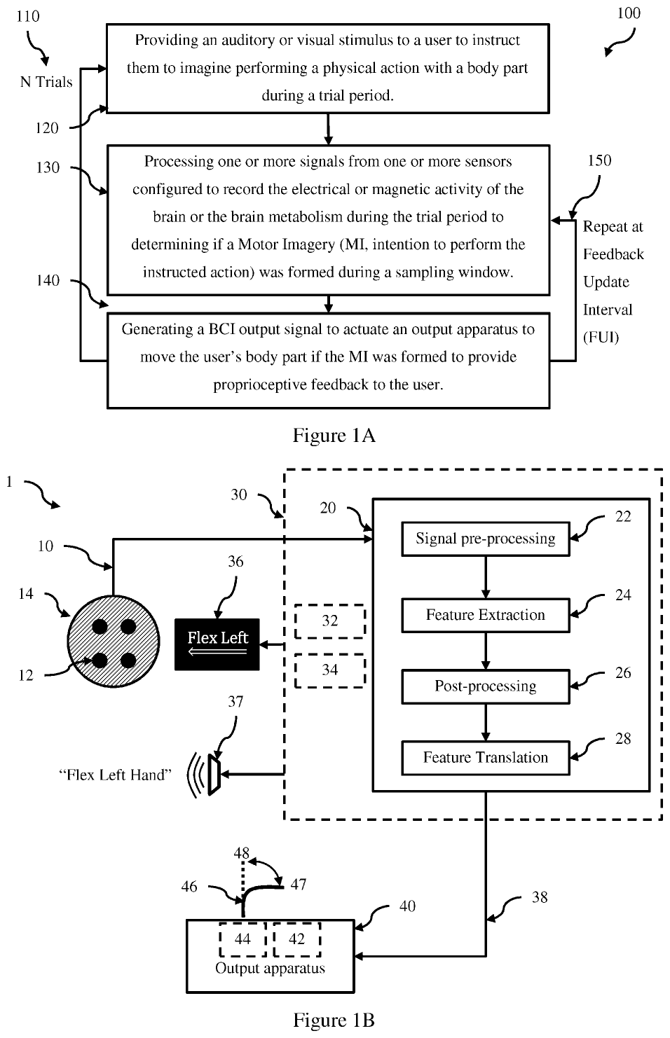

Referring now to FIGS. 1A and 1B, there is shown a flowchart of a Motor Imagery (MI) based Brain-Computer Interface (BCI) rehabilitation method 100, and a MI based BCI rehabilitation system 1 according to an embodiment. Embodiments of the method and system focus on providing more optimal feedback provision (compared to prior art systems) by using a Feedback Update Interval (FUI) of 100 ms or less over a course of treatment. Further embodiments use reaction time measurements to select optimal FUI values for patients, as well as gradually reducing the FUI value over the course of the treatment. Reaction time measurements of the unaffected limb are used to determine the lower limit of the FUI value, and reaction time measurements of the affected limb are used to determine the initial FUI value. Optimisation of FUI has not been previously been studied, and prior studies on the use of BCI for stroke rehabilitation using real-time proprioceptive feedback have typically used FUIs in the 200-300 ms range. In contrast the present method uses much shorter FUI values of less than 100 ms and as will be outlined below has produced a clinically significant improvement over the course of treatment. Whilst not be bound by theory it is believed that by providing repeated feedback at an appropriate feedback rate of 100 ms or less, the proprioceptive signals arrive at neurons in the brain at an appropriate feedback rate to lead to Hebbian (neuroplastic) reinforcement of neurons leading to improved motor control and rehabilitation outcomes. That is, in embodiments of the present system feedback movements are provided a sufficient rate to reinforce motor neural connections leading to improved patient outcomes.

The rehabilitation method 100 is performed as a series of trials 110 using a brain-Computer Interface (BCI) based rehabilitation system. The plurality of trials may be broken into a plurality of sessions where each session comprises a plurality of trial runs, and each trial run comprises a set of consecutive trials using the same FUI value. A trial 110 begins by providing an auditory (ie oral) or visual stimulus to a user to instruct them to imagine (but not actually try) performing a physical action with a body part during a trial period 100. In the context of this specification the body part will be considered to be a limb or a part of a limb such as a hand. For example as shown in FIG. 1B, a computer 30 comprising a memory 32 and a processor 34 sends output to a display screen 36 such as “Flex Left” and an arrow pointing to the left to instruct a user of the system (the patient) to imagine flexing their left hand. Additionally or alternatively a speaker 37 may broadcast an audio cue such as the phrase “Flex Left Hand”. In this embodiment the body part is a hand, but the system could be used for other body parts (ie limbs or parts of limbs) such as fingers, wrist, forearm, elbow, upper arm, toes, foot, ankle, lower leg, knee, upper leg etc. The trial period may be a short length of time such as under 10 seconds (eg 3, 4 or 5 seconds).

The system is configured to generate one or more BCI input signals 14 from one or more sensors 12 configured to record the electrical or magnetic activity of the brain or the brain metabolism during the trial period which are processed by a computing device 30. At step 130 the BCI input signals 14 from the sensor 12 are processed 20 by processor 34 to determine if a Motor Imagery (MI, ie an intention to perform the instructed action) was formed during a sampling window (during the trial period). In one embodiment, a plurality of electroencephalography (EEG) sensors 12 is fitted to the user using a wearable apparatus, for example via a skull cap 14 or headset worn by the user. The output signals 10 of the EEG sensor are sent to the computing apparatus 30, typically via an amplifier stage (not shown). The computing apparatus may comprise additional electronic modules or boards to perform signal conditioning or pre-processing, sample and hold, and signal processing. The computing apparatus may include a chargeable battery.

BCI processing code 20 comprises instructions to perform one or more of signal pre-processing 22, feature extraction 24, post-processing 26 and feature translation 28. At step 140 a BCI output signal 38 is sent to actuate an output apparatus 40 to move the user's body part if a motor imagery of the body part was formed (as determined in step 130) to provide proprioceptive feedback to the user (ie signals from nerves in the user's muscles and skeletal system informing them of movement of the body part). The output apparatus 40 may comprise a microprocessor (or similar controller) or similar computing apparatus 42 and one or more electronically controlled motor such as a servomotor or stepper motor 44 which move (actuate) a flexible orthosis or body part support 46 on which the body part either rests (ie supported by) or which the body part is attached (eg strapped in place). For example in this embodiment the orthosis/body part support 46 is a flexible plate to which supports a hand which is strapped to the plate. The plate is moved by servomotors 44 between a flexed position 47 and an extended position 48 in a series of incremental steps.

The process 20 of detection a motor imagery of a body part 130, and generating an output to move the body part 140 is periodically (ie repeatedly) performed during the trial period at a Feedback Update Interval (FUI) 150. Embodiments of the method and system use an FUI of 100 ms or less, and processing the BCI input signals is performed in a time less than the FUI. Thus as the user continues to think about movement of the body part, the BCI detects this MI intention (via the BCI input signals) and incrementally moves the body part. Over the course of the trial period the user thus receives continued proprioceptive feedback (driven by the output apparatus moving the body part).

In one embodiment the plurality of trials 200 are broken into a plurality of sessions (220, 230 and 240) spaced apart in time (ie by days or weeks), where each session comprises a plurality of trials (eg N trials). The individual trials (240ₙ) within a session 240 may be grouped into trial runs comprising consecutive trials. Rest periods may be included between trial runs and between individual trials with a trial run (and in some embodiments may be relaxation trials). During the course of treatment, the FUI value may change, and in some embodiments the FUI value may start at a value larger than 100 ms and then after some number of sessions the FUI is decreased until it is below 100 ms. The FUI may be incrementally decreased. Further the FUI values may be temporarily increased during the course of treatment (for example if performance declines), provided it is later decreased until it is eventually below 100 ms. For example the sequence of FUI value could start at 700 ms, 300 ms, 100 ms, 96 ms, 48 ms, and 16 ms. In other cases the sequence of FUI values could be 300 ms, 100 ms, 75 ms, 150 ms, 75 ms, 50 ms and 25 ms. In another case the FUI sequences could be 96 ms, 48 ms, 32 ms, and 16 ms. Other sequences are possible provided the FUI values eventually drop below 100 ms.

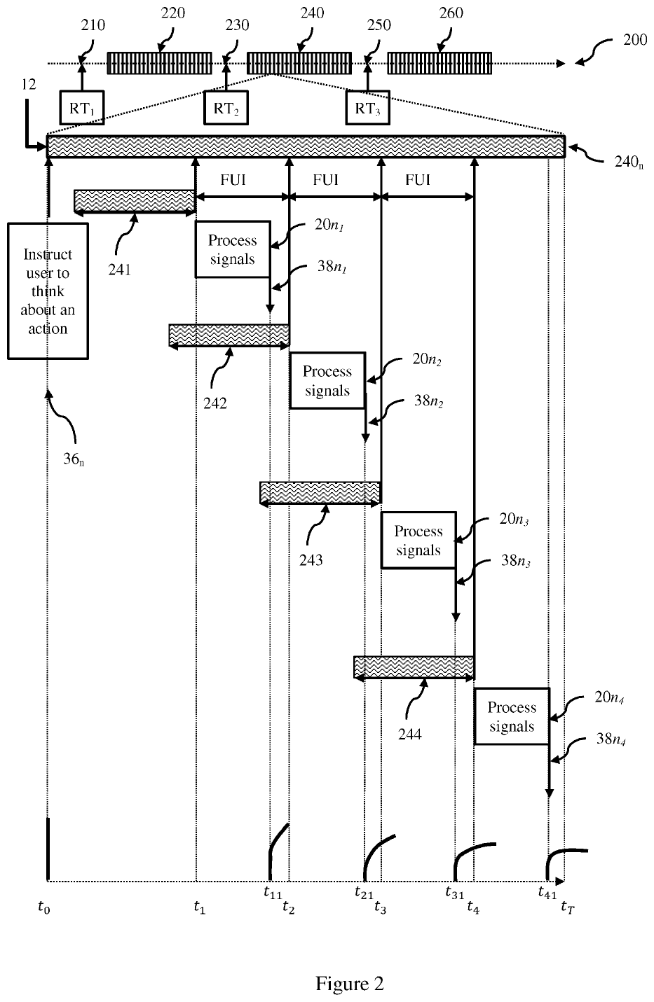

To further illustrate the method FIG. 2 is a schematic diagram of trial sequence 200 of a BCI based rehabilitation method 100 according to an embodiment. An individual trial 240ₙ comprises receiving BCI input signals 12 over the trial period of length T (t₀, t_{T}). At the start of the trial (t₀) the user's hand is in an extended position and the user receives a visual or auditory instruction to think about performing a body action 36ₙ such as flexing the user's hand to a flexed position. Then at time t₁ the BCI system begins processing 20ₙ₁ a sample 241 of BCI input signals collected just prior to (or up to) time t₁ and an output signal 38ₙ₁ is sent to output apparatus at time t₁₁ (assuming a motor imagery was detected) to incrementally move the user's hand. Then at time t₂ after a FUI interval from t₁ (ie t₂=t₁+FUI), the BCI system begins processing 20ₙ₂ another sample 242 of BCI input signals collected just prior to (or up to) time t₂ and an output signal 38ₙ₂ is sent to output apparatus at time t₂₂ (again assuming a motor imagery was detected) to incrementally move the user's hand. In this example this is repeated twice more before the end of the trial. That is at time t₃ after a FUI interval from t₂ (ie t₃=t₂+FUI=t₁+2*FUI), the BCI system begins processing 20ₙ₃ another sample 243 of BCI input signals collected just prior to (or up to) time t₃ and an output signal 38ₙ₃ is sent to output apparatus at time t₃₃ (again assuming a motor imagery was detected) to incrementally move the user's hand, and at time t₄ after a FUI interval from t₃ (ie t₄=t₃+FUI=t₁+2*FUI), the BCI system begins processing 20ₙ₄ another sample 244 of BCI input signals collected just prior to (or up to) time t₄ and an output signal 38ₙ₄ is sent to output apparatus at time t₂₂ (again assuming a motor imagery was detected) to incrementally move the user's hand to the flexed position.

Various sensors can be used to generate signals that reflect electrical/magnetic activity of the brain or the brain metabolism, and can thus be used as BCI input signals. These include electrodes to record Local Field Potentials (LFP) within the brain's cortex, mainly represent the cortical activity within 0.1-1 mm of the recording electrode(s). These signals provide high quality and signal-to-noise ratio, however it requires surgery to mount the electrodes within the brain. Electrocorticogram (ECoG) signals are recorded from the surface of the brain cortical tissues and are known to reflect the cortical activity within 2-5 mm around the recording electrodes. Here, ECoG electrodes are mounted on the surface of the brain through small holes made in the skull and thus are less invasive than LFPs. The ECoG also provides a reasonable quality and signal-to-noise ratio signal. Electroencephalogram (EEG) signals are obtained from the scalp where each electrode represents the neural activity of 10-40 cm ² of the cortical sheet centred around the electrode. The biggest drawback of EEG is its low signal-to-noise ratio that renders it prone to environmental and biological artefacts. However EEG is the only a non-invasive signal that measures the brain electrical activity, it is the preferred sensor and input signal for BCI systems (such as embodiments of the present system).

The magnetoencephalogram (MEG) records the brain's very small magnetic fields. This technology that was introduced in the 1980s, and is more sensitive than EEG to cortical activities oriented parallel to MEG sensors. Another advantage of MEG over EEG originates from transparency of the skull and other tissues circumscribing the brain to the magnetic field. However, in practice, EEG electrodes are placed much closer to the neural sources than MEG sensors. However MEG equipment is significantly more costly than EEG hardware, requiring a magnetically shielded chamber, and its sensors need to be cooled using liquid helium.

Measuring the brain activity through its electromagnetic activity has a number of inherent limitations such as reflecting the activity only in the immediate vicinity of the sensors. The brain's metabolic processes, however, provide an overall picture of the whole brain via its energy consumption that is correlated with neuronal firing rate. Increased brain metabolism is reflected by greater consumption of required resources such as sugar and oxygen that necessitate increased blood flow in the brain. The blood flow in the brain is locally controlled and thus can be employed as a biomarker for neural activity. Four main technologies to monitor the brain metabolism are summarized as follows.
Functional transcranial Doppler (fTCD) is method that measures the brain metabolism via monitoring changes in blood flow through the brain's main arteries. Its advantages comprise affordability and mobility of the equipment. However, it only measures differences between the right and left hemispheres activity that renders it suboptimal for BCI studies.
Positron emission tomography (PET) tracks the blood flow through tracing injected radioactive compounds. Similar to fTCD, PET is not an attractive option for BCI research as it is relatively slow and radioactive compound injection is an invasive technique.
Functional near infrared spectroscopy (fNIRS) monitors the blood flow in the brain through screening modification in different types of the haemoglobin cells. These changes in haemoglobin types are referred to as blood oxygen level dependent (BOLD) response. It measures the response of haemoglobin cells exposed to a near infrared radiation to calculate the blood flow accordingly. Its spatial resolution is on the order of centimetres and has a temporal resolution on the order of several seconds.
Functional magnetic resonance imaging (fMRI) is another technique that like fNIRS also monitors the BOLD response to measure the brain metabolism. It screens the response of different haemoglobin cells to a magnetic field to determine oxygen consumption and blood flow. Whilst it presently is the most sensitive technique for monitoring the brain metabolism with high resolution it is also the most costly.

Embodiments of the present method and system preferably use EEG sensors/signals as the input signals for the BCI system as they are relatively low cost, non-invasive and easily applied to a patient, and are capable of non-invasive measuring the brain electrical activity of the patient. However in other embodiments other sensors (such as those discussed above) could be used to generate input signals to determine if a MI (intention to perform the instructed action) was formed during a sampling window.

The BCI system is configured to detect the signature of motor functions in BCI input signals (as both intended movement and actual movement produce similar signatures). Movement-related cortical potentials (MRCP) can be extracted by averaging EEG signals (and other BCI input signals) recorded over the sensorimotor cortex before and after a voluntary movement. The MRCP begin with a slowly increasing negativity, referred to as readiness potential or Bereitschaftspotential (BP). Next, they continue with a larger negativity starting about 400 ms prior to the start of movement or motor imagery, named as negativity slope (NS). The initial slope of the motor potential occurs just before the onset of electromyographic (EMG) activity and is topographically focal over the primary motor cortex (M1) that may reflex the activation of M1. The mentioned focal negativity lasts for 30-50 ms following the onset of EMG activity. Next, the peak negativity moves toward the anterior contralateral area, where it reaches its largest negativity, known as the frontal peak of motor potential. There is evidence that motor imagery evokes similar MRCPs to those of motor actions. Whilst evoked potentials in imagery exhibits lower and delayed peaks compared to MRCP with actual movement, MRCP related responses in the primary and supplementary motor areas are similar for motor imagery and motor actions to allow a BCI system to detect a signature of intended motor functions in input BCI signals (such as EEG signals).

Prior to, during, and after motor actions and motor imagery the oscillatory patterns of BCI input signals, such as EEG signals, recorded over the sensorimotor cortex change. These alterations in the oscillatory activity of the sensorimotor cortex are divided into two main subcategories Event Related Desynchronization (ERD) and Event Related Desynchronization (ERS).

Processing motor and cognitive tasks prior to and during motor actions and motor imagery poses a high demand on these cortical areas. Also, receiving sensory information during motor execution is an additional processing task for the sensorimotor cortex. As a result different neuronal ensembles within the sensorimotor cortex attend to different tasks and consequently, a desynchronization occurs among them that are manifested as lower amplitude in the oscillatory activity of this region. This phenomenon is known as event related desynchronization (ERD). Therefore, ERD is considered as a signature of the cortical activation for motor action and motor imagery. After the offset of motor action or imagery, activated neuronal networks have no more processing task and thereby return to their baseline (idling) status. Subsequently, a synchronisation occurs that is known as event related synchronization (ERS). The ERS is used as a signature of baseline activity (idling status) for the EEG signals recorded over the sensorimotor cortex. The ERD/ERS phenomena are thought to occur as a result of thalamo-cortical and cortico-cortical feedback loops. They are time locked but not phase-locked, ie they have different manifestations for different frequencies. The frequency band of interest that is usually used for ERD/ERS analysis of EEG signals lies within 0-50 Hz in which few specific frequency bands exist: i) Delta δ band (0-4 Hz); ii) Theta θ band (4-8 Hz); iii) Alpha α band (8-13 Hz); iv) Beta β band (16-30 Hz); v) lower Gamma γ band (30-50 Hz). Among the mentioned frequency bands, Alpha and Beta bands are more explored within the MI-BCI studies and so are preferably used as the selected frequency bands to be processed. However it is to be understood that other frequency bands could also be used to detect motor imagery intentions. As ERD occurs both prior to and during motor imagery performance, the present MI based BCI method and system preferably detect ERD related signals from the sensorimotor cortex using the one or more BCI input signals (eg EEG signals) as indicating to detect a motor imagery of a body part by the user, and focus on applying appropriate feedback to the user to reinforce ERD signals. However in other embodiments other appropriate BCI input signals (ie from other sensors) or frequency bands could be used, including combinations of signals (eg MCRP, ERD, and ERS event detection).

To summarise, and whilst not being bound by theory, it is hypothesized that using a specific BCI design that provides appropriately timed and recurrent sensory feedback provides the basis for the occurrence of i) Hebbian plasticity that not only increases ERD and eases BCI training, may also result in behavioural gains for stroke survivors; and ii) operant learning through perception of late responses to the sensory stimuli. The synergy between the Hebbian plasticity and operant conditioning not only shortens the training time for ERD modulation (maintaining patient involvement/motivation), but also enhances the efficacy of restorative BCI.

In one embodiment determining if a MI (intention to perform the instructed action) was formed during a sampling window comprises pre-processing the one or more signals to reduce noise and/or artefacts, performing feature extraction on the pre-processed one or more signals, post-processing extracted features to improve feature distribution and/or mitigate redundancy, and using a feature translator to determine if a MI (intention to perform the instructed action) was formed during the sampling window.

Signal pre-processing 22 involves artefact removal and enhancement of spatial, temporal and/or spectral characteristics of the EEG signals using a priori knowledge. Pre-processing may include one or more of pre-filtering, down sampling, spatial filtering, artefact removal-environmental noise, and artefact removal biological noise. These are briefly discussed below.

Pre-filtering: frequency range pre-filtering removes unwanted frequencies while retaining desired frequencies. For instance, in motor imagery based BCIs (MI-BCI) sensorimotor rhythms (8-30 Hz) are explored to extract spectral features and therefore, frequency range pre-filtering for MI-BCI involves band-pass filtering of EEG signals to eliminate spectral features outside 8-30 Hz frequency band.

Down Sampling: To enhance computational efficiency, the data are down sampled following the primary filtering by a suitable factor. The adjusted sampling rate must remain at least two times greater than the largest frequency of interest in the signal. This approach guarantees preservation of all relevant information in the signal through keeping the sampling frequency greater than Nyquist frequency and thereby prevent signal distortion.

Spatial Filtering: EEG signals reflect the activity of a fairly large area of the brain. Furthermore, the recorded signals of the channels close to each other are largely correlated. To enhance the spatial resolution of EEG electrodes and to render them more representative of independent cortical activities a myriad of spatial filtering techniques have been proposed. Data dependent spatial filtering encompasses principal component analysis (PCA), independent component analysis (ICA), and common spatial patterns (CSP). It increases the spatial resolution while considering the covariance between all available channels. However, data independent spatial filtering such as with the Laplacian filter and common average reference (CAR) do not deal with the relationship among different electrodes.

Artefact Removal—Environmental Noise: Environmental artefact removal involves removing electrical and magnetic interference originated from the environment out of EEG signals. For instance, the power-line noise (50 Hz in Europe, Asia and Australia or 60 Hz in America) interfere with EEG signals. Power-line noise is produced when the electromagnetic fields of the equipment working at 50/60 Hz affect the human body that produces strong noise in their respected frequencies. To remove this artefact a band-stop (notch filter) is applied to EEG signals.

Artefact Removal—Biological Noise: Biological noise that affects EEG signals mainly manifests as muscular contraction potentials, ie electromyogram (EMG), and eye movement potentials, ie electrooculogram (EOG) and eye blinks Since these signals affect the neighbour electrodes similarly, spatial filtering techniques such as Laplacian filtering remove a large portion of them.

Following pre-processing that suppresses the noise and enhances desired aspects of the signals, the next step is to extract relevant features using a priori knowledge of the recorded signal. Notably, for motor imagery-based BCIs, the desired features are amplitude modulation of EEG signals within 8-30 Hz frequency band. To extract these features Fast Fourier Transform, Autogressive modelling or wavelet transform may be performed:

Fast Fourier Transform: The fast Fourier transform (FFT) is a computationally efficient method used to calculate the spectral power of a signal within an individual frequency or a frequency band. The FFT takes an N sample signal and produces N frequency samples uniformly spaced over −f/2 to +f/2 range where f represents the sampling frequency. Note that FFT values are complex values that have magnitude and phase. To calculate the spectral power, we square the magnitude of FFT values. To reduce artefacts introduced by abruptly changing edges that occur through segmentation of the EEG signals, tapering windows such as Hamming and Hanning window functions are multiplied by the finite-length signals prior to FFT calculation. This approach mitigates the occurrence of unwanted ripples in the frequency response.

Autogressive modelling: The autoregressive (AR) modelling technique is an alternative to the Fourier transform in calculating spectral attributes of EEG signals. It models EEG as output of a filter that receives white noise as its input. Since white noise encompasses all frequencies, the filter adjusts its parameters to reflect EEG spectral characteristics. A critical factor in AR modelling is optimal order determination. Note that EEG signals are considered to encompass up to five spectral peaks comprising δ (0-4 Hz), θ (4-8 Hz), α (8-13 Hz), β (16-30 Hz), and lower γ (30-50 Hz). Therefore, the order of the AR model must be more than 10 for proper modelling of EEG signals. However, the order must be increased when large sampling rates are used. The Burg algorithm is a preferred method for calculating AR model parameters as it guarantees model stability. The power spectrum is obtained using the formula 1:

$${P}_{A R} ( ω ) = \frac{{E}_{p}}{{\vert 1 + {\sum }_{k = 1}^{p} {a}_{p} ( k ) {e}^{- j k w} \vert}^{2}} \qquad ( 1 )$$

where Eₚ is the prediction error, αₚ(k) is the kₜₕ filter weight, p is the order of the AR model, and P_{AR}(ω) is the spectral power at the angular frequency of ω.

Wavelet Transform: To overcome the drawbacks of FFT and AR modelling that lose temporal information when transforming a signal to the frequency domain, wavelet transform has been proposed. Wavelet analysis convolves the EEG signal with both stretched and compressed versions of specifically shaped wavelets. Whenever it finds a large correlation, it creates larger wavelet coefficients and vice versa. Therefore, not only do wavelets reveal spectral attributes of the EEG signal, but they also disclose how the spectral behaviour of the signal changes over time. Another aspect of a wavelet transformation is that it adjusts the signal window length in order to maximize its frequency resolution. Notably, by applying longer wavelets for low frequencies and shorter wavelets for high frequencies, it extracts frequency domain features with optimal resolution. The wavelet transform has two versions: (i) a more computationally efficient version is discrete wavelet transform (DWT) that minimizes redundancy, and (ii) the continuous wavelet transform (CWT), which is more robust in extracting subtle features.

Prior to transferring the extracted features to classifiers, further processing (post-processing) of features is required to optimize feature distribution and mitigate redundancy among features. Post-processing of features is typically carried out through the following procedures to enhance the performance, accuracy, and speed of the classifier.

Normalisation: The extracted feature sets may have different means and dynamic ranges, non-related to the BCI task conditions. In such cases, a normalization procedure is applied. It involves subtracting the average value from all features, followed by dividing the resultants by the standard deviation. This technique renders zero mean and unit variance features that enhance classification outcomes.

Log-Normal transformation: The FFT amplitude lower range is bounded by zero whereas the higher range is limited by the sampling frequency. In addition, EEG power is inversely proportional to frequency. Thus, it is very likely to produce a non-Gaussian distribution of spectral power features with EEG signals. Most classifiers provide optimum results when receiving normally distributed features. A logarithmic transformation in most cases normalizes the non-normal EEG spectral features, and makes them optimal for classification.

Dimension Reduction: To provide real time feedback in a BCI framework, it is necessary to keep the computational cost as low as possible. To decrease computational cost of the classification, it is required to extract the lowest possible number of features that represent EEG signals. In addition, representing the EEG signals with larger than optimal number of features causes the curse of dimensionality and degrades the classification accuracy with novel observations. Principal component analysis (PCA) and independent component analysis (ICA) are among the most commonly applied methods that remove correlated and redundant features from the feature space and define the optimal features.

In some embodiments the extracted features of BCI signals directly reflect a subject's intent. However in some embodiments a feature translator is used to transform the features into signals amenable to external devices.

A feature translator, that is also referred to as classifier, is a mathematical model that includes a number of parameters. The parameters of the model become adjusted using observations in which the subject's intent is clear. Then the adjusted (trained) model is used to predict the subject's intention from new observation data (generalization). To determine a high-performance classifier for a specific BCI system, its model type, input features, and parameters need to be selected optimally. The following describes these aspects of BCI classifiers.

The primary function of a classification model is to determine whether the user's intention is reflected in the extracted features. The goodness-of-fit of a selected model will be subsequently determined through the accuracy, and speed of classification. Model selection depends mostly on two critical factors: BCI application, and the amount of available training data.

Classifiers that produce continuous outputs have a regression model (ie linear regression based models) whereas those with discrete outputs have a discriminant model (ie linear discriminant analysis). While models are typically used in a specific manner to provide either discrete or continuous outputs, they are usually able to produce both types of outputs. A number of most commonly used classifier models for BCIs are listed as follows:

Linear Least-Squares Discriminant Classifier: This model is one the simplest and meanwhile most powerful models used for BCI classification. Its general form is:

Y=b₁X₁+b₂X₂+ . . . +bₙXₙ+a (2)

where Y is the predicted value (classifier output), b₁, b₂, b₃, . . . , bₙ and a are the model weights that need to be determined, and X₁, X₂, X₃, . . . , Xₙ are features. The bᵢ parameters are defined using the following formula:

b=(X′X)⁻¹X′Y. (3)

Bayesian classifier: This classifier uses the maximum likelihood concept to extract information from a priori knowledge to classify novel data as posterior probabilities. More specifically, it calculates the possibility of belonging to each class of outputs given a set of features. The class with the highest possibility is most likely the class that the novel observation belongs to. The general form of a Bayesian classifier is as follows:

$$P ( Y \vert {X}_{1} , {X}_{2} , … , {X}_{n} ) = \frac{P ( Y ) P ( {X}_{1} \vert Y ) P ( {X}_{2} \vert Y ) … P ( {X}_{n} \vert Y )}{P ( {X}_{1} ) P ( {X}_{2} ) … P ( {X}_{n} )} \qquad ( 4 )$$

where Y is the predicted value (classifier output), X₁, X₂, X₃, . . . , Xₙ are features, and P(Xᵢ|Y) is the probability of Xᵢ given Y.

A Bayesian classifier is simple and robust even when a small number of observations for classifier training is available. However, for a large set of training data, a linear least-squares discriminant model that takes into account the relationship among features may provide a better performance

Support Vector Machines (SVM): The aforementioned classifiers originate from statistical methods. However for another family of classifiers such as support vector machines, machine learning approaches are employed to iteratively improve the classifier performance. Notably, support vector machines find the support vectors across the boundaries between classes that: i) minimize the Euclidean distance between the support vectors (hyper planes) and incorrectly classified data; ii) maximize the Euclidean distance between support vectors. This model is believed to be robust against outliers and generalizes well, even with limited number of observations for adjusting the parameters.

Non-linear methods: Advanced machine learning approaches transform non-linear problems into linear ones through application of kernel methods. Commonly used kernels include Gaussian and radial basis function (RBF) kernels. Gaussian kernels allow producing fairly distinctive hyperplanes from features that in their original form would have created very irregular shapes.

Another method to deal with feature non-linearity is to exploit the robustness of artificial neural networks that are able to estimate (theoretically) any function, where a sufficient number of artificial neurons have been used. For example an adaptive neuro-fuzzy inference system (ANFIS) classifier can be used. However, complex functions necessitate employing a large number of artificial neurons that require large numbers of observations to fine tune the model parameters for artificial neural networks.

Regardless of feature types used for BCI classification, be it event-related potentials or spectral power, the brain usually produces correlated (redundant) features. In addition, the number of features used in any model is proportional to its classification accuracy of the training data. However, the model generalizability degrades when very large number of features are used for parameter adjustment as it causes overfitting to the training data. Therefore, a trade-off must be made between minimizing the error of the classifier with training data, and its generalizability through selecting optimal number of features. Heuristic methods, iteratively select the optimum features that fulfil the mentioned trade-off. Step-wise heuristic methods such as forward and backward stepwise heuristic techniques, instead of examining all possible combination of features, start with the best features and add or subtract other features and check whether they make the classifier more accurate in each step. The search for optimal features continues until a stopping criterion such as reaching a minimum r² value, that defines the percentage of correct outputs that can be predicted given the selected features, is fulfilled.

Parameters of a model can be estimated using direct methods such as the LDA Equation 2 or using an iterative optimization algorithm. The latter approach has the advantage of being able to estimate even non-linear systems, while the former method is fast and computationally efficient. A common technique in parameter estimation is the least-mean squares (LMS) algorithm in which using Equation 5, the parameters are adjusted and iteratively decrease the estimation error. Here, b(t+1) is the updated parameter at time t+1, l is the learning-rate parameter, e(t) is the prediction error, and X(t) is the current feature vector

b(t+1)=b(t)+l·e(t)·X(t). (5)

The LMS algorithm belongs to a family of parameter estimators referred to as adaptive parameterization methods. They perform well when the relationship between the error, features, and class labels are simple. If the relationship is complex, LMS may not provide an optimal solution. In such cases evolutionary algorithm such as Genetic Algorithms, and Particle Swarm Optimization may perform better than LMS as they are robust against falling into local minima and sub-optimal final points.

Various method exist for classifier evaluation such as accuracy, sensitivity, selectivity, and specificity. In a typical BCI application, depending on the subject's intention and the BCI output command, four scenarios might occur. To describe these conditions we use a typical BCI system that translates neural activity to commands for an exoskeleton that opens the subject's fingers:

Scenario 1: The user aims to open his fingers and the BCI opens his fingers; this is referred to as true positive (TP)

Scenario 2: The user does not aim to open his fingers and the BCI does not open them; this is referred to as true negative (TN)

Scenario 3: The user does not aim to open his fingers but the BCI opens them; this is referred to as false positive (FP)

Scenario 4: The user aims to open his fingers but the BCI does not open his fingers; this is referred to as false negative (FN).

The most commonly used BCI measure is accuracy that in terms of the aforementioned indices is:

$$accuracy = \frac{T P + T N}{T P + T N + F P + F N} \qquad ( 6 )$$

Other BCI measures including sensitivity, selectivity (positive predictive value), and specificity (negative predictive value) are defined as follows:

$$\begin{matrix}sensitivity = \frac{T P}{T P + F N} & ( 7 ) \\ selectivity = \frac{T P}{T P + F P} & ( 8 ) \\ specificity = \frac{T N}{T N + F N} . & ( 9 )\end{matrix}$$

There is no specific measure that is suitable for all BCI applications and it is the attributes of the BCI system that makes one or a specific collection of the mentioned metrics more suitable than others.

For BCIs with continuous outputs such as cursor position updates on monitors, the mentioned measures may not be suitable and other specific measures are required. One such possibility is the Chi-squared (χ²) measure that sums the squared errors. This measure reflects the output variance that is caused by the BCI error (1−r²). Then, r² indicates the model's goodness-of-fit that can be calculated.

Information Transfer Rate (ITR) is a measure that accounts for the speed and accuracy of a BCI system simultaneously. This measure calculates the amount of information units transferred per unit time and is calculated using the following formula:

$$B = {log}_{2} N + P {log}_{2} P + ( 1 - P ) {log}_{2} \frac{1 - P}{N - 1} \qquad ( 10 )$$

where B is the bit-rate, N represent the number of classes, and P is the accuracy of the classifier.

In one embodiment pre-processing is performed using an 8-30 Hz band-pass filter and a spatial filter, feature extraction is performed using an autoregressive (AR) model with at least 10 orders and the feature translator performs classification of the coefficients of the AR model using a support vector machine to classify whether an event related desynchronization (ERD) in the 16-30 Hz (β) band occurred during the sample window.

Efficient performance of BCI systems typically involves a trade-off between the sample window length, classification accuracy and classifier update rate (FUI). Such a trade-off ensures that real time feedback is provided with the highest possible accuracy at an appropriate rate to facilitate Hebbian learning. A feasible trade-off for these parameters was investigate by using a dataset provided by the University of Graz, Austria available at http://www.bbci.de/competition/iv/. In this study a continuous wavelet transform and the Student t-test for feature extraction and an SVM for classification. To compare the accuracy of the classification for different window lengths, we defined the window length of the training and test data to be 250, 500, 750, or 1000 ms and then shifted the time windows in steps of 32 ms.

The accuracy level for time windows 250 and 500 ms started with classification accuracies very close to chance, however, for wider time windows (750, 1000 ms), the classifiers begin with accuracies higher than 60%. Moreover, with all time windows, after almost one second the classification reaches its maximum and that maximum level has a direct relationship with the length of the time window. A comparison of the average values and standard deviations of the classification accuracies for 250 ms, 500 ms, 750 ms and 1000 ms time windows demonstrated a direct correlation between time window lengths and the average accuracy values and an inverse correlation between time window lengths and accuracy values' standard deviations. Table 1 below shows classification statistics for different window lengths. This table compares the mean, standard deviation, rise time (the time that it takes to reach to the maximum accuracy), maximum, and last accuracies of different time windows with 250, 500, 750, and 1000 ms length for the subject-independent classifier.

TABLE 1
Classification statistics for different window lengths. This table
compares the mean, standard deviation, rise time (the time that
it takes to reach to the maximum accuracy), maximum, and last
accuracies of different time windows with 250, 500, 750, and
1000 ms length for a subject-independent classifier.

| Window | First | Rise | Max. | Last | Mean | |
|---|---|---|---|---|---|---|
| Length | Accuracy | Time | Accuracy | Accuracy | Accuracy | Std. |
| (ms) | (%) | (ms) | (%) | (%) | (%) | Deviation |
| 250 | 49 | 1000 | 67 | 60 | 61.09 | 3.8 |
| 500 | 54 | 900 | 71 | 62 | 64.06 | 3.3 |
| 750 | 61 | 1000 | 72.3 | 62 | 66.45 | 2.7 |
| 1000 | 65 | 750 | 72.9 | 64 | 68.16 | 2.5 |

The 750 ms and 1000 ms time windows provide maximal average accuracies with smallest standard deviations. Thus in some embodiments the sampling window is between 500 ms and 2000 ms and ends just prior to the start of processing the one or more signals. In some embodiments the sampling window is between 750 ms and 1000 ms (inclusive).

In one embodiment the hardware comprises a 64-channel Waveguard EEG cap connected to a 72-channel Refa TMSi EXG amplifier, containing 64 unipolar and eight bipolar channels. In one embodiment EEG data were recorded from 8 channels (F3, F4, T7, C3, Cz, C4, T8, Pz) positioned according to the international 10-20 system of electrode placement. The AFz channel was used as the ground channel based on the recommendation of the manufacturer. The impedance between electrodes and the scalp was kept below 50 k and this is sufficient due to the amplifier input impedance in the order of tera-Ohms. The amplifier does not require a reference channel as it uses built-in common average referencing of the recorded channels. It also disregards any electrodes with very high impedance (more than 256 k) and excludes them from the common average reference. The signals were digitized at 1024 Hz and were then passed through a 50 Hz notch filter (3rd order Chebyshev) followed by a band pass filter (1st order Butterworth) with corner frequencies set to 0.1 and 49 Hz. The EEG input signals were provided to a computer running BCI2000 software modified to supply auditory commands and concurrently update servomotor position of orthosis during feedback. However custom software could be used to process the BCI input signals as described herein.

In some embodiments, the BCI output signal is a binary signal, and the output apparatus incrementally moves the body part if a first signal is received, and the output apparatus does not moves the body part if a second signal is received. In one embodiment, the body part is a hand, and the output apparatus 40 comprises a microcontroller 42 which controls a servomotor 44 to control the position of a flexible orthosis 46 configured to support a hand and move fingers from a fully flexed position to a fully extended position in a series of incremental steps. Suitable micro controllers comprise a Micro Maestro servo controller module that can control up to 6 servomotors. Suitable servomotors include Blue Bird BMS-630 which receive comments from a controller and incrementally adjust the angle of the orthoses. In one embodiment the orthosis comprises a mechanical body made of PVC that supports participants' hands in all assumed situations (from fully flexed fingers to fully extended fingers). The orthosis is attached to a support using a modular base that allows adjustment of the orthosis angles (tilt and pan) in all directions.

To update the flexion angle of the orthosis with proprioceptive feedback, for every feedback update, a BCI output control signal binary value as a go/no go signal to change or not change the orthosis angle. That is the BCI output signal was a normalized value that represents current status of the oscillatory cortical signals, an indicated whether ERD had occurred. The amount of increment may be determined based on the length of the trial period and the feedback update interval to ensure that the orthosis does not reach the maximum deflection prior to the end of the trial period, so that the user receives feedback for the trial period.

Feedback optimization is believed to enhance the performance of motor imagery based brain-computer interfaces (MI-BCI). Many studies have investigated the effect of feedback modality on MI-BCIs. However, the impact of feedback update interval (FUI) modification on MI-BCI performance remains unexplored. We hypothesized that: (i) information processing speed and BCI aptitude are correlated; and, (ii) the FUI modification affects BCI performance depending on subjects' information processing speed. To further optimise MI-BCI a study on the effect of FUI change on ten healthy participants who were dichotomized as good and poor imagers according to their online BCI accuracy. They attended two BCI training sessions with 16 ms and 96 ms FUIs. Simple reaction time (SRT) measure that was recorded as an index of information processing speed was found to be a surrogate for BCI aptitude with 70% accuracy. Also, it was illustrated that for good imagers 16 ms FUI provided higher BCI accuracies whereas poor imagers showed better performance with 96 ms FUI. Overall, this preliminary study suggests that FUI customization may improve the performance of MI-BCIs.

Ten healthy participants (six males) aged 18-26 years were recruited in this study. A 72 Channel Refa TMSi EXG amplifier, with 64 unipolar and eight bipolar channels and a 64 channel Waveguard EEG cap were used. The EEG data were recorded only from small Laplacian combination of the channels centred on either C3 or C4 channel. The ground channel was connected to the participants target hand using a wristband. The impedance between electrodes and the scalp was kept below 20 k. The EMG data of the finger flexor muscles of the target hand was recorded using a bipolar channel of the EXG amplifier. The amplifier uses a built-in common average reference (CAR) of the recorded channels and thus does not require a reference channel. The amplifier excludes any unipolar channels with impedances more than 256 k from the common average reference calculation. It also does not consider the bipolar channel used for EMG recording in common average referencing of EEG signals. All EEG and EMG signals were digitized at 1000 Hz and then were passed through a 50 Hz notch filter (3rd order Chebyshev) followed by a high pass filter (1st order Butterworth) with corner frequency set to 0.1 Hz.

To provide the proprioceptive feedback two orthoses were mounted on a platform to serve either the right or the left hand. They passively flexed fingers of the involved hand according to the motor imagery of the target hand. Each orthosis was driven by a Blue Bird BMS-630 servomotor. The commands to operate the orthoses originated from customised software and then using a Micro Maestro servo controller module were translated to the servomotors. A customized version of the BCI2000 CursorTask was used to record the data and run the online experiments. The source code was customized to provide auditory commands and to update the position of the servo motors.

Participants performed MI-BCI and received proprioceptive feedback that updated every 16 ms and every 96 ms, respectively. We assumed that any potential effect of FUI alteration becomes clear when in one condition feedback is updated six times faster than the other. In the rest of the specification, for simplicity, FUIs of 96 and 16 ms will be regarded as “long FUI” and “short FUI”, respectively.

Each online session comprised eight runs of MI of right/left hand finger flexion. Each run included 20 trials with ten motor imagery and ten relaxation trials presented with a randomized order. Each run took almost four minutes, and consecutive runs had a 2-minute break in between where each recording took less than an hour. Sessions were scheduled using BCI2000 Operator scripts that determined runs operation and the breaks between consecutive runs.

Every trial started with a “start” auditory command that prepared the participant for the following instruction. After 3 s, another auditory command instructed the participant to either relax or perform motor imagery of right hand finger flexion. After another 3 s, the participant was able to receive contingent feedback according to their motor imagery or relaxation as follows. For “right” auditory command the right orthosis briskly initialized the right hand's fingers to fully extended position. Then within the next 2.5 s, the orthosis was able to flex the right hand's fingers incrementally if motor imagery classification result was smaller than a threshold value. However, if the command was “relax”, the free running left orthosis was immediately initialized to fully extended position. Then it could be flexed incrementally within the next 2.5 seconds if the classification result was larger than a threshold value. Note that the threshold value is defined according to the pooled average spectral power of motor imagery and relaxation trials within the most recent 18 seconds. Next, an auditory ‘stop’ command cued the end of each trial and after a subsequent 4 s inter-trial interval, the next trial started. Note that each participant's left hand was on the arm rest and not placed on the left orthosis. As a result, participants received proprioceptive feedback for right hand imagery and visual feedback through observation of the left orthosis flexion on relaxation. For participant P3, however, his left hand was involved with the left orthosis while his right hand was resting on the armrest. Thus, participant P3 was supplied with proprioceptive feedback for left hand imagery and visual feedback of relaxation through the right orthosis.

We employed one trial-based index and one run-based index to compare the participants' online BCI performance with different FUIs. The indices are as follows: (i) A trial based information transfer rate (ITR) was employed to take into account both the accuracy and the speed of data transfer. The ITR calculation was performed according to Equation 11 in which it is expressed in bits per minute (bits/min)

$$ITR = {log}_{2} N + P {log}_{2} P + ( 1 - P ) {log}_{2} ( \frac{1 - P}{N - 1} ) ( \frac{60}{8.5} ) \qquad ( 11 )$$

where P stands for the accuracy of each trial, N represents the number of classes (two classes: relaxation and motor imagery), and 8.5 is the total length of each trial in seconds. The ITR has been multiplied by 60, to express it in bits/min. Note that the trial-based accuracy (P) was calculated as the percentage of times in the feedback section of each trial that classification outputs conformed to the task and flexed the orthosis. (ii) A run based classification accuracy that indicates the percentage of trials in each run in which the average classifier output is larger or smaller than the threshold value for the motor imagery and relaxation trials, respectively. While the former measure (ITR) considers both the average accuracy and speed of information transfer across all trials, the latter is equivalent to the traditional measure of BCI performance; the target hit rate in each run with visual feedback. The analysis was performed using custom built Matlab scripts. It has been shown that the threshold for BCI accuracy to consider one is controlling a BCI is 70%. Therefore, in this study participants were dichotomized to good imagers if their average accuracy with the short FUI were more than 70%, and poor imagers if their average accuracy with the short FUI were between 50% and 70%.

A simple reaction time (SRT) test was carried out, using the CANTAB battery test of Cambridge Cognition, to measure the reaction time of participants. Participants sat in a chair and were asked to concentrate on a tablet computer placed on a desk in front of them and to press the button on a press pad as soon as they saw a square on the screen. Each participant performed the task 30 times to obtain the average latency (reaction time), which was used as their SRT index. Note that our reported values for SRT are measured using CANTAB battery test of Cambridge Cognition with subjects aged 18-26 years and using different hardware, software, or participants with different age range may provide different results.

For offline analysis of the EEG and EMG signals, EEGLAB and custom-built Matlab scripts were used. The EEG signals were spatially transformed using a small Laplacian filter to produce single channel EEG data with enhanced spatial resolution (C4-SLP for P3 and C3-SLP for other participants). Next, the data were band-pass filtered (3-47 Hz) and divided into epochs from −2 to 8.5 s centred around the “start” auditory command Then all relaxation trials were rejected and after removing the average baseline, data purification was performed as follows: (i) to tag the outlier trials, EEG amplitude, spectral power, skewness, kurtosis and variance were checked; (ii) the trial was labelled as irregular if any of the mentioned indices were beyond the regular values of artefact free EEG signals using the guideline provided elsewhere. The EMG signals of the Flexor Carpi Radialis (FCR) of the target hand, which monitored the reflection of actual movement of fingers in the forearm muscle activity, were also band-pass filtered (3-400 Hz). Next, the average baselines of motor imagery trials were removed and then they were epoched using the same time windows as EEG signals. EMG signals recorded during the motor imagery performance were screened, and trials with peak-to-peak values larger than 50 mV were tagged. All tagged trials due to irregular EEG or significant EMG signals were discarded (9.2%).

The spectral power of the feedback section of motor imagery trials (6-8 s) and their preceding inter-trial interval (−2 to 0 s) were extracted in three frequency bands: α (8-13 Hz) and lower β (16-22 Hz) and higher β (22-30 Hz). Only the last 2 s of the 4-second-long inter-trial interval were considered as baseline period. This adjustment ensured that the post imagery ERS had elapsed and had not affected the baseline spectral power estimation. Only the first 2 s of motor imagery with feedback section (6-8 s) was considered, to equalize the length of imagery and baseline time windows. Welch method with frequency resolution of 0.25 Hz was used to estimate the power spectral density (PSD) in decibel (dB). The PSD in the inter-trial interval preceding the imagery trials was also calculated to determine baseline spectral power. The absolute difference between the spectral power during motor imagery and inter-trial interval were calculated as a measure of absolute ERD for each frequency band. The ERD percentage indices were then calculated according to Equation 12:

ERD(%)=(A−R)/R×100 (12)

where A and R stand for the spectral power during motor imagery and the baseline period, respectively. Note that ERD percentage measures in each frequency bands were calculated and compared between different FUIs in each group of imagers (good and poor imagers).

To investigate the relationship among the online accuracy measures and SRT, a Pearson correlation analysis between SRT and the accuracy was carried out for each FUI. The accuracy and ITR indices of the eight runs of each session with either short or long FUI for each participant were used, to calculate the online BCI performance measures. Since each group (good and poor imagers) had five members, each condition (FUI) comprised 40 (five participants×eight runs) measures for comparison. We decided to consider all eight runs of each session for each participant's accuracy and ITR measures (instead of their average values) to increase the statistical power. A two-way ANOVA with factors BCI aptitude (levels “good” and “poor”) and FUI (levels “16 ms” and “96 ms”) was used to explore the interplay between the aforementioned factors and the accuracy and ITR, separately.

In the offline analysis, α, lower β and higher β ERDs were compared. The calculations were performed for each of eight runs of each session with either short or long FUI for all participants. Selecting all runs for the analysis, resulted in 40 (five participants×eight runs) ERD measures with each FUI in each frequency band for each group. In total, it provided 240 ERD measures (40 runs×two FUIs ×three frequency bands) that were analysed using a two-way ANOVA with factors frequency band (levels α, lower β, and higher β) and FUI (levels “16 ms” and “96 ms”) for good and poor imagers, separately.

For post-hoc tests in the applied ANOVA for online measures, since two correlated measures (accuracy and ITR) were used, only planned comparisons between FUI values (16 and 96 ms) were carried out. Therefore, Holm-Sidak's two-sided t-test was adopted for post-hoc analysis to adjust for multiple comparisons. However, in the two-way ANOVA on ERD measures, since spectral analysis in α and β bands are independent, no adjustment for multiple comparisons were made and thus, uncorrected Fisher's LSD two-sided t-test was adopted for post-hoc analysis.

Table 2 summarizes the online accuracies and ITR values of all participants at short and long FUIs and their SRT results. Our reported results for SRT test corroborate with findings of another study by philip1999simple who found the SRT values for subjects less than 30 years old as 236±32 ms. Subjects with accuracies >70% at an FUI of 16 ms (P2, P3, P6, P9, P10) were grouped as good imagers. The remaining participants (P1, P4, P5, P7, and P8) who achieved accuracies between 50% and 70% at the same FUI were grouped as poor imagers. We observed a linear relation between the accuracy and SRT (FIG. 0). The Pearson correlation coefficient between SRT and accuracy at an FUI of 16 ms was r=−0.671 (p=0.033) while its value between SRT and accuracy at an FUI of 96 ms was r=−0.725 (p=0.018). Bootstrapping the samples for FUI of 16 ms resulted in r′=−0.671±0.140 with 95% confidence interval of (−0.371-−0.889) between SRT and the accuracy. For FUI of 96 ms bootstrapping resulted in r′=−0.7165±0.1654 with 95% confidence interval of (−0.346-−0.957) between SRT and accuracy. Since none of the calculated 95% confidence intervals contained zero, bootstrapping further demonstrated the significance of the observed correlations. It suggests that SRT is a surrogate for BCI aptitude with both long and short FUIs. The green horizontal lines in FIG. 0 represent the accuracy threshold of 70% that has been used to classify participants as good and poor imagers. As illustrated, the boundary margin for classification is wider at 16 ms FUI compared to that of 96 ms FUI. Therefore, the accuracy with the short FUI was employed to dichotomize subjects as good and poor imagers.

TABLE 2
Summarizing SRT, ITR and accuracy results

| | Accuracy | Accuracy | ITR (16) | ITR (96) | SRT |
|---|---|---|---|---|---|
| Participants | (16) | (96) | (bits/min) | (bits/min) | (ms) |
| P1 | 53 | 59 | 3.15 | 3.45 | 244 |
| P2 | 86 | 80 | 4.68 | 4.80 | 206 |
| P3 | 84 | 76 | 4.60 | 4.28 | 214 |
| P4 | 59 | 69 | 3.33 | 4.05 | 230 |
| P5 | 67 | 61 | 3.44 | 4.40 | 245 |
| P6 | 97 | 86 | 6.95 | 4.65 | 214 |
| P7 | 56 | 57 | 3.20 | 3.15 | 221 |
| P8 | 64 | 81 | 4.17 | 4.98 | 219 |
| P9 | 89 | 90 | 6.13 | 5.26 | 221 |
| P10 | 83 | 91 | 4.12 | 4.45 | 208 |

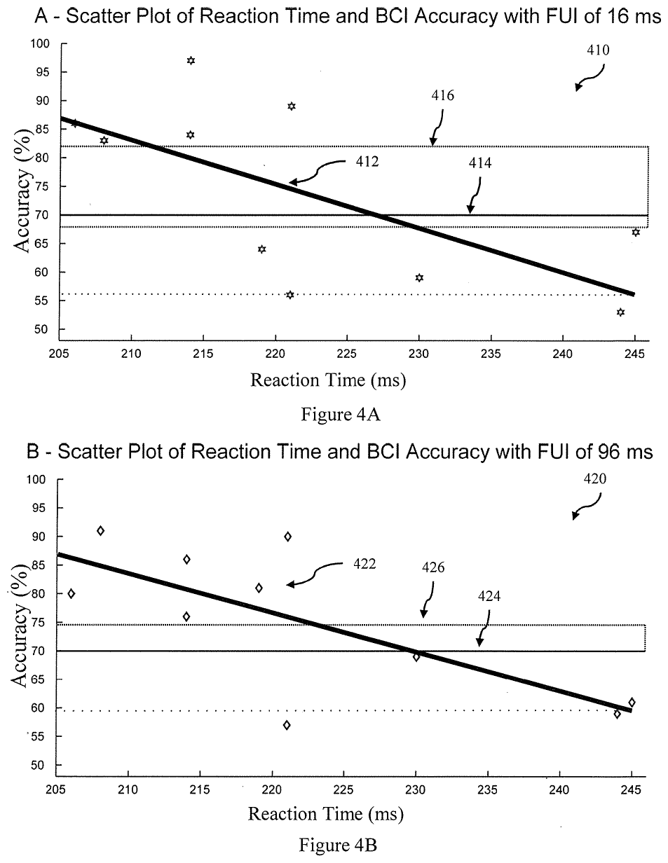

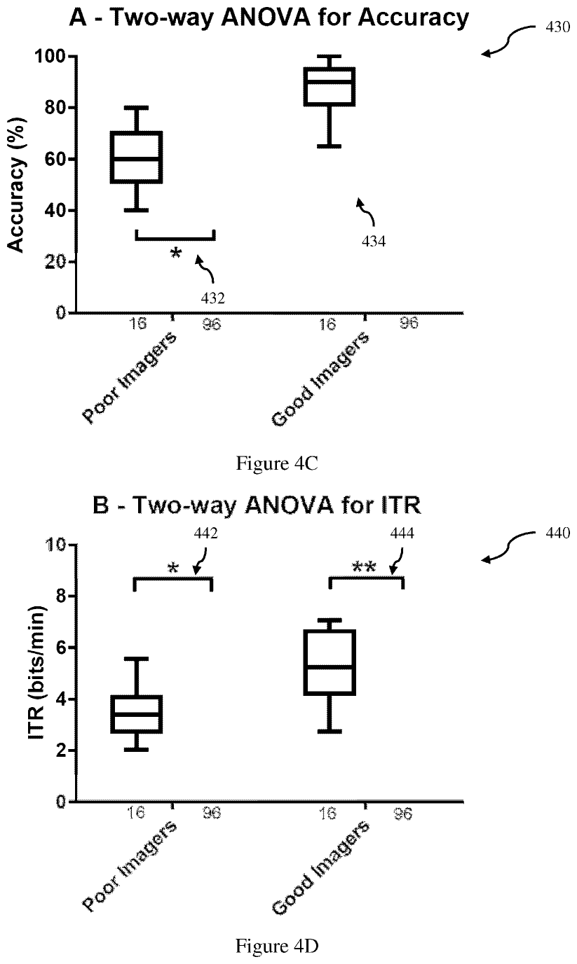

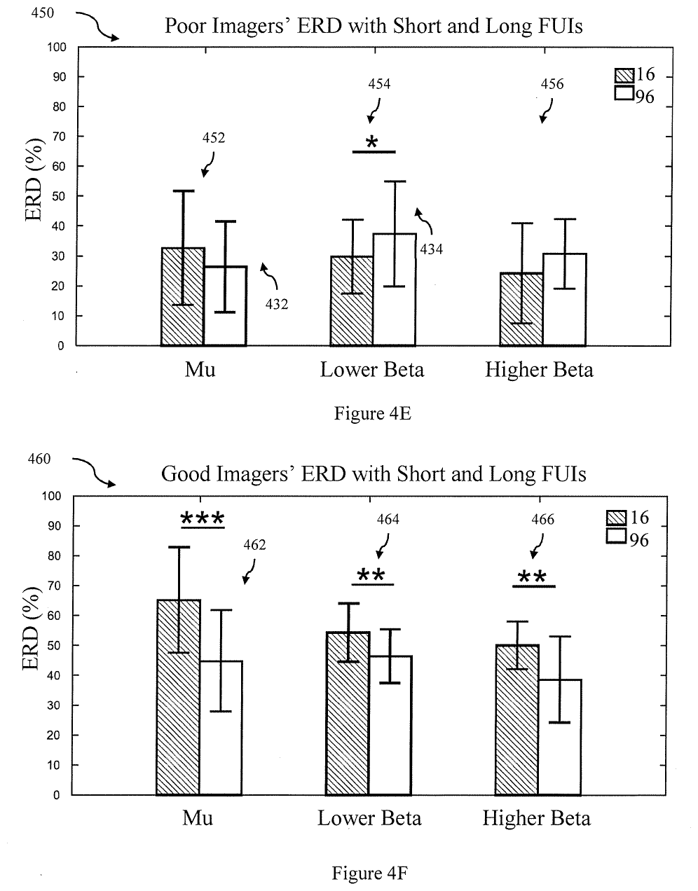

FIG. 4A is a scatter plot of reaction time and BCI accuracy with a FUI of 16 ms 410 according to an embodiment and FIG. 4B is a scatter plot of reaction time and BCI accuracy with a FUI of 96 ms 420 according to an embodiment. The red and blue linear regression lines for 16 ms 412 and 96 ms 422 conditions, are shown in FIGS. 4A and 4B respectively. The green horizontal lines 414 424 at 70% accuracy were used to segment subjects as good and poor imagers. Since the boundary margin (the green shadowed area around the green line) 416 in FIG. 4A was wider than that of boundary margin 426 in FIG. 4B, the online accuracy with 16 ms FUI was adopted to label participants as good and poor imagers.

To analyse the online BCI performance, the run-based accuracy and trial based ITR were used to compare the effect of long and short FUIs on good and poor imagers, separately. FIG. 4C is a two way ANOVA plot 420 for BCI accuracy for a group of poor imagers and a group of good imager at FUIs of 16 ms and 96 ms according to an embodiment. FIG. 4D is a two way ANOVA plot 440 of BCI Information Transfer Rates for a group of poor imagers and a group of good imager at FUIs of 16 ms and 96 ms according to an embodiment. According to FIGS. 4C and 4D and, the direction of accuracy and ITR change following FUI modification was dependent on the BCI aptitude. Two-way ANOVA of the accuracy showed significant main effects for BCI aptitude (F(1,78)=172.2, p<0.0001) but not for FUI (F(1,78)=0.6704, p=0.4154), with a significant interaction (F(1,78)=8.212, p=0.0053). The post-hoc analysis showed that for poor imagers the short FUI improved the accuracy significantly compared to the long FUI (t(78)=2.605, p=0.0219). However, changing FUI provided no significant difference between accuracies for good imagers (t(78)=1.447, p=0.1518). The two-way ANOVA for the ITR showed a significant interaction between two factors (F(1,78)=17.80, p<0.0001) and a significant main effect for BCI aptitude (F(1,78)=38.16, p<0.0001). However, FUI factor did not have a significant main effect (F(1,78)=0.4037, p=0.5270). The post-hoc analysis showed a significant outperformance of the short over long FUI for good imagers (t(78)=3.432, p=0.0019). In contrast, for poor Imagers ITR was larger with long FUI than those of short FUI (t(78)=2.534, p=0.0264). Overall, poor imagers appear to produce larger accuracies and ITRs with long FUI, whereas good imagers revealed larger ITRs with short FUI. Furthermore, there was a highly significant main effect of BCI aptitude with both the accuracy and ITR across good and poor imagers.

Power spectral density of feedback section of motor imagery trials and the baseline period preceding imagery trials were calculated for all participants and averaged in both groups in the 3-45 Hz frequency band. For each FUI in each group the difference between the spectral power of motor imagery and baseline periods were calculated. Also, ERD percentage measures for both groups and both condition in α, lower β and higher β frequency bands were calculated according to Equation 12 and are demonstrated in FIGS. 4E and 4F. FIG. 4E is a plot 430 of poor imagers' Event Related Desynchronization (ERD) percentages across different ECG frequency bands for FUIs of 16 ms and 96 ms according to an embodiment. FIG. 4F is a plot 440 of good imagers' Event Related Desynchronization (ERD) percentages across different ECG frequency bands for FUIs of 16 ms and 96 ms according to an embodiment (*: p<0.05, **: p<0.01, ***: p<0.001).

The statistical analysis was performed on the ERD percentages as the neural signature of increased cortical activity during motor imagery performance. The ERD indices were analysed using two-way way ANOVA with factors frequency bands (levels α, lower β, and higher β) and FUI (levels “16 ms” and “96 ms”) across good and poor imagers, separately. Two-way ANOVA of good imagers revealed significant main effects for both frequency band (F(2,234)=6.178, p=0.0024) and FUI (F(2,234)=32.06, p<0.0001). The post-hoc analysis with Holm-Sidak's two-sided t-test showed a significant outperformance for 16 ms FUI over 96 ms in the α (t(234)=4.155, p<0.0001), lower β (t(234)=2.896, p=0.0041), and higher β (t(234)=2.757, p=0.0063) bands. However, for poor imagers, there was not any significant main effects neither for frequency band (F(2,234)=2.563, p=0.0792) nor for FUI (F(2,234)=1.647, p=0.2007). However, there was a significant interaction between fastors (F(2,234)=3.343, p=0.0370). The post-hoc analysis for poor imagers showed that lower β band supplied significantly larger ERDs with the long compared to those of the short FUI (t(234)=2.036, p=0.0428) . Overall, good imagers showed significantly stronger ERDs across all frequency bands with the short FUI while poor imagers showed significantly larger ERDs at lower β band with the long FUI.

The main findings of this study are as follows: (i) the SRT and BCI aptitude measures are inversely correlated, ie a short SRT is a surrogate for possessing a high-level BCI aptitude and vice versa; (ii) the FUI customization affects the BCI accuracy, ITR, and down-regulation of sensorimotor rhythms when operating MI-BCIs with proprioceptive feedback depending on the participants' level of BCI aptitude. Notably, participants with poor BCI aptitude produce higher accuracies and larger ERDs with feedback updated every 96 ms, while good imagers provide a higher ITRs and elicit stronger ERDs with feedback updated every 16 ms.

We hypothesized that FUI affects BCI performance differently depending on subjects' reaction time. SRT was found to be inversely correlated with BCI accuracy at both short and long FUIs. Moreover, the slope of the regression line fitted between SRT and the accuracy for 96 ms FUI showed to shift slightly lower for good imagers and more clearly higher for poor imagers. If increasing FUI had a symmetric effect on good and poor imagers, we should have observed a symmetric shift in the slope of the regression line of the 96 ms FUI. Thus, it appears that increasing FUI from 16 to 96 ms improved BCI performance for poor imagers, but degraded the performance of good imagers to a lesser extent. Accordingly, it may suggest that people who possess a high level of BCI aptitude benefit more from shorter FUIs. However, longer FUIs do not degrade their BCI performance significantly. In contrast, poor imagers with a lower BCI aptitude appear to respond positively to FUI prolongation.

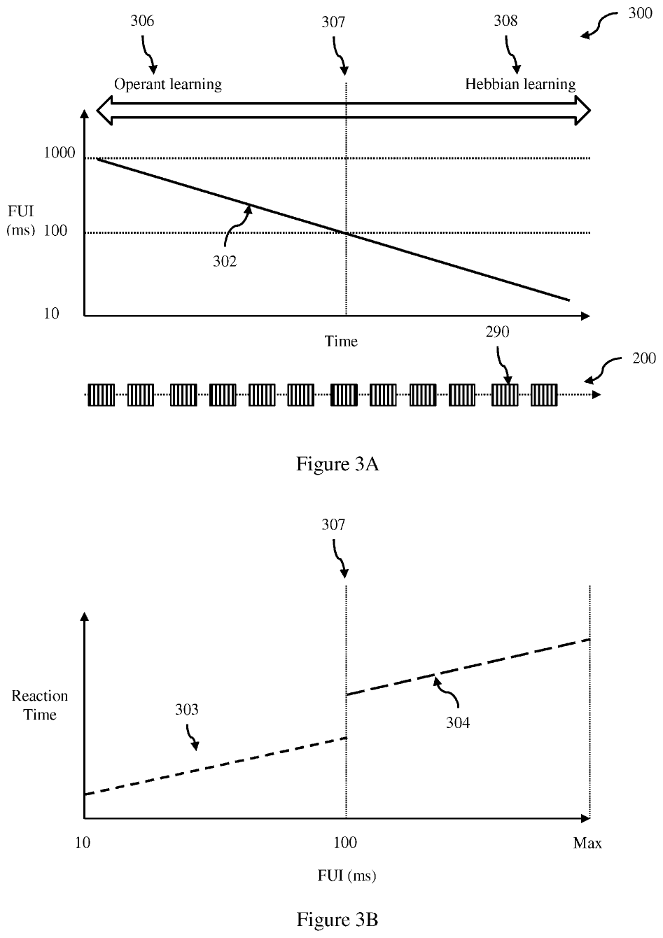

Thus the above study indicated that participants could be categorized as poor and good imagers according to their online BCI accuracy with FUI of 16 ms, and this was correlated with their reaction times. It is hypothesised that poor imager with slow reaction times are unable to process feedback at small FUI and thus have less optimal outcomes. Thus a measurement of the reaction time of the unaffected limb can be used to set the lower limit (smallest) FUI value for an user. Thus in some embodiment the plurality of trials 200 are broken into a plurality of sessions 290, each session comprising a plurality of trials. The method then further comprises the steps of measuring a reaction time of an unaffected limb of the user prior to performing one or more trials, as illustrated in FIG. 2 in which reaction times 210, 230, and 250 are measured prior to trials (or sessions) 220, 240, and 260 respectively. The FUI interval for the one or more trials is then based upon the measured reaction time on the basis that reaction times are positively correlated with FUI values such that shorter reaction times generate shorter FUIs. For example FIG. 3A is a schematic plot showing the change in Feedback Update Interval (FUI) over a series of trials according to an embodiment. In this plot, the FUI value is plotted on the left y axis and time is plotted on the x axis. The solid line 302 indicates the FUI value used over time during a plurality of sessions 290 as part of a course of treatment 200. FIG. 3B shows the correlation between FUI and reaction time. In this figure he dashed line 303 represents the reaction time of the unaffected limb of the patient and the minimum FUI value associated with the reaction time, which in this embodiment is linear and shows a positive correlation (ie low or fast reaction times are associated with low minimum FUI values). Thus prior to each session, the reaction time could be measured and mapped to a FUI value using a mapping function or classifier, such as one based on calibration trials or previous research. In some embodiments determining the FUI for the one or more trials comprises classifying the reaction time into one of a plurality of reaction time ranges where each reaction time range has an associated FUI value.

In some embodiments the same FUI value is used for all trials (ie a reaction time measurement is made, and the FUI selected for all trials based on the reaction time measurement). In other embodiments the FUI value is decreased from an initial FUI value to a minimum FUI value where the minimum value is determined (or set) based upon the reaction time measurement of the unaffected limb. Reductions may be based on measured progress using a measure of improvement. In some embodiments the FUI value may be increased in a course of treatment if the user performance drops before being decreased again (ie the decrease is not strictly progressive, and could bounce between values). Multiple reaction time measurements could be taken during the course of treatment, and the minimum could be reduced if the reaction time improves.

Reaction times measurement of the affected limb can also be used to determine the initial FUI value at the start of the course of treatment. Such reaction time measurements are an indication of the degree of impairment of the patient. Subsequent reductions in the FUI may be based on a measure of improvement or measurements of the reaction time of the affected limb. In some embodiments the method further comprises the step of measuring the reaction time of affected limb the individual and if the reaction time is greater than a first threshold 370, then the FUI interval is initially set to an initial value between 100 ms and an upper value such as 1000 ms, and the FUI is progressively reduced or shortened over a series of sessions until FUI is less than 100 ms. In the case that there is no residual motor function in the affected limb, and thus a reaction time measurement cannot be obtained, then the FUI value is set to the maximum value. Repeated measurements of the reaction time of the affected limb may be taken during the course of treatment and used to progressively reduce the FUI value. A measure of improvement may be obtained after one or more sessions and if this exceeds some threshold then FUI is reduced until the FUI value is less than 100 ms. At that point a measurement of the reaction time of the unimpaired limb could be used to determine the minimum FUI value to be used. In FIG. 3B long dash line 304 represents the reaction time of the affected limb of the patient and the associated FUI value, which in this embodiment is linear and shows a positive correlation (ie slow or large reaction times are associated with large initial FUI values.

Various reaction time measurements may be obtained. In some embodiments the reaction time measurement may be a simple reaction time measurement (SRT), in which a single stimulus is provided to the user and the time taken to respond to the stimulus recorded. In other embodiments a choice reaction time measurement (CRT) could be used, in which a number of stimuli are provided to the user. In each case the modality of the stimulus/stimuli may be visual, auditory, haptic, proprioceptive, or any combination thereof.

In one embodiment the reaction time may be calculated as the difference between the time of stimulus/stimuli exposure and the onset of observing a significant increase in the amplitude of an electromyogram (EMG) signal of the target muscle or a significant change in the area under curve of the absolute or squared EMG signal within a time window of up to 500 ms. In another method, for those patients with residual motor functions, they may be asked to push/release a button (for SRT) or one of the multiple buttons (for CRT) when they receive the stimulus/stimuli. In another method, the patient may wear specific rings on each finger, each of which may be vibrated or electrically stimulated, and the patient is asked to move the finger in the ring that is vibrated or electrically stimulated. Reaction time measurements can be taken off both the affected limb and the unaffected limb and can be used to provide measures of stroke recovery (ie improvement) and to determine when to change the FUI and by how much.

As will be outlined below, and without being bound by theory, it is believed that for some patients, their initial impairment prevents Hebbian learning, and thus initial trials seek to improve patient capability through operant learning 306 (ie repeatedly performing an action). The FUI is progressively decreased and once reaction times drop below a threshold 307, the FUI is dropped to below 100 ms to enable Hebbian learning 308 to occur. As outlined above, a measure of improvement may be obtained during or after a session (or a trial) and used to determine whether to reduce the FUI in a subsequent session (or trial). In one embodiment if the measure of improvement exceeds a threshold (eg indicating significant improvement) then the FUI value is reduced. A range of measures of improvement can be used. As outlined above, reaction time measurements of the affected limb could be taken of the course of treatment (eg after each session, or every 5 sessions, or every week, etc), and this could be compared with the initial value to determine absolute improvement, or with recent measurements to determine relative improvements. Reaction time measurements of the unaffected limb could be used as control measurements.

In another embodiment the measure of improvement may be based on accuracy of performing a task. For example during a trial run, the number of times that the user was able to achieve some threshold level of movement, such as movement of their limb from the flexed to extended positon using motor imagery, could be counted. If this exceeds a threshold (eg 75%) then the FUI value is decreased.

In another embodiment the level of Event Related Desynchronisation (ERD) could be used to obtain a measure of improvement. In this embodiment, consecutive trials within a run are interspersed with relaxation trials during which the user does not imagine moving the body part. ERD times are recorded during the MI trials and relaxation trials within a run and the measure of improvement (ERD) is calculated based on the difference between the spectral power of the MI trials and the relaxation trials during the run. In one embodiment the ITR (equation 11) could be calculated using the MI trials and relaxation trials as the two groups and in another embodiment the ERD percentage difference for MI vs relaxation trials (equation 12) could be used.

In some embodiments the relaxation trials are simply time when the user does nothing, or is asked to do nothing, or to meditate such as focusing on breathing. In other embodiments the relaxation trials similar to MI trials are performed in which a user watches an orthosis (or other feedback output device) move based on detected relaxation. Additionally or alternatively auditory feedback may be provided. Signals from sensors configured to record the electrical or magnetic activity of the brain or the brain metabolism during the trial period at a FUI (eg EEG signals) are processed. The processing is performed to determine if a MI (intention to perform the instructed action) was formed during a sampling window. A BCI output signal is generated provide visual feedback via the output apparatus if relaxation was formed. For example in one embodiment an identical orthosis (without involving any body parts) is in view of the user, and gets extended recurrently during the relaxation trial if it is detected or determined that the relaxation task was performed. Equation 11 or 12 can then be used to determine the measure of improvement and compared with a threshold value (eg obtained through calibration or other trials). The number of successful relaxation trials could also be used as a measure of accuracy.

In another embodiment the measure of improvement comprises taking a plurality of measurements of an active motor evoked potential (MEP) of the user. This may comprise obtaining a plurality of measurements of active MEP, wherein each measurement comprises providing an auditory stimulus to a user to instruct them to extend a finger and then measuring the amount of finger extension force. For example a strain gauge based system could be used in which the force is transformed to a voltage by a strain gauge integrated into the device and then a sample and hold circuit conditions the measured signal and transfer it to a PC or any other processor such as an embedded system. Feedback is provided to the user indicating the amount of applied force and if the measured finger extension force is within a desired range. In one embodiment a software program on the PC or the processing device uses the measured signal to guide the patient for production of the expected amount of force by showing them a visual real-time feedback on a screen where a cursor moving vertically (updated in real-time) and the desired region is bounded between two horizontal lines. In one embodiment the desired range is between 10-10000 grams of force and more preferably 10-1000 grams of force, and the optimal range for a patient may be selected based on the specific condition (degree of impairment) of the patient.

As soon as the amount of force falls within the expected (or desired) range, the program triggers the transracial magnetic stimulation machine to stimulate the target muscle. Simultaneously, it triggers the EMG amplifier to start recording and presents the recorded active motor evoked potential (MEP) on the screen. Next it calculates the peak-to-trough value of the active MEP signal and saves it as the MEP of that specific trial. An estimate of the average active MEP using the plurality of the peak-to-trough values can then be obtained. Robust estimators may be used (median, trimmed means, weighted means, etc.). In one embodiment the average active MEP of each session is defined as the average of 15 consecutive trials. In some embodiments active MEP, and changes in MEP may be used as alternatives to reaction time measurements for determining the FUI or when to change the FUI. In some embodiments the active MEP may be used with reaction time measurements.

A study was performed to test the effect of shorter than usual feedback update intervals affect behavioural and neurophysiologic measures following BCI training for stroke patients. The ARAT score that was used as the primary and behavioural measure showed an unprecedented (more than 30%) increase over the course of training, while neurophysiologic measures including MEP and MVC showed distinctive changes in early and late phases of BCI training.

Inclusion criteria: (1) being in the chronic stable phase of stroke—at least six months post-stroke; (2) having impaired motor capabilities in their arms—by screening them using action research arm test (ARAT) scores to be less than 54 out of 57; 3) having intact cognitive functions—by screening them using mini-mental state examination (MMSE) score to be more than 26 out of 30; (4) being independently mobile—with or without a walking aid; (5) not having any transcranial magnetic stimulation (TMS) contraindications by screening them using TMS adult safety screen (TASS) questionnaire; (6) not having excessive tone in their arm and hand muscles—by screening them using modified Ashworth test score to be less than three out of four; (7) having the ability to perform vivid motor imagery—by screening their accuracy in running a motor imagery based BCI system to be more than 70%; (8) having an intact sense of proprioception—by screening their blind judgement of comparing size of seven polystyrene balls with more than 90% accuracy. Results for a patient who was a 65-year old man and had a stroke in his right hemisphere 3.5 years prior will now be presented.

In this study we planned to investigate: (i) whether and how neurofeedback training with FUIs selected from 16-96 ms range (16, 24, 48, or 96 ms) affects the motor performance, and (ii) in case of observing any potential effect of these shorter than usual FUI values on behavioural and/or neurophysiological measures, how long the impacts last. Therefore, we made a specific setup that not only recoded the performance measurements during neurofeedback training sessions; it also measured the indices up to 5 weeks after BCI training interventions.

To fulfil the aforementioned goals, the study was designed as a proof-of-principle study with a ABABCC setup. The selected participant took part in a number of index measurements (IM) in no intervention weeks (A), intervention weeks (B), and follow-up weeks (C). In the no intervention weeks (A), only the IM were done on Monday, Wednesday and Friday. However, in intervention weeks (B), besides the measurement of indices similar to week (A), on every weekday a BCI training session was also performed. Then, in the follow-up weeks (C) performance indices were measured once per week, to investigate how long potential changes last, one and five weeks after the last neurofeedback training session (weeks 5, and 9).

The study was of nine weeks duration and was set as ABABCC. In A weeks (weeks 1 and 3), only performance measures were recorded three times per week. In B weeks (weeks 2 and 4), in addition to recording performance measures three times per week, five neurofeedback sessions were carried out where the FUI value for each session are shown in braces. In C weeks (weeks 5 and 9), only one recording of performance measures was performed. During weeks 6-8, no recording sessions were performed. In weeks 2 and 4, all measures were recorded after BCI training (IM: index measurement, BCI: neurofeedback training session).

A 72 channel Refa TMSi EXG amplifier, containing 64 unipolar and eight bipolar channels and a 64 channel Waveguard EEG cap were used for data acquisition. A screening session indicated an optimum frequency of 15 Hz with a large Laplacian configuration of EEG channels centred on CP4 channel produced the highest coefficient of determination (r²) for the left hand motor imagery versus relaxation trials.

To cope with real-time constraints of the BCI system with very short FUIs such as 16 ms, only five out of 64 EEG channels (FC4, CPz, CP4, PO4 and TP8) were used to record EEG signals during training sessions. The AFz channel was used as the ground channel. Also, one bipolar channel was used to record EMG activity from the extensor digitorum communis (EDC) muscle to monitor a potential voluntary finger extension during MI. The impedance between the scalp and recording electrodes were kept below 10 kΩ. The amplifier uses a built-in common average referencing algorithm where any electrode with high impedance is excluded from common average reference calculation. The sampling frequency was set to 1000 Hz. To remove DC offset and non-related high-frequency elements, a bandpass filter with corner frequencies set to 0.1 and 48 Hz was also applied. The BCI2000 was adopted as the software platform where we customized it to supply auditory commands and concurrently update servomotors' position throughout the feedback section of each trial.

To provide proprioceptive feedback, two orthoses (one for each hand) to passively extend four fingers. The left orthosis was associated with the participant's affected (left) hand to provided proprioceptive feedback during MI. The right orthosis, which was not involved with the patient's hand, provide visual feedback via observation of the orthosis extension during relaxation trials. Each orthosis position was controlled via a servomotor (Blue Bird BMS-630). A Micro Maestro servo controller module translated BCI2000 commands and accordingly operated the servomotors.

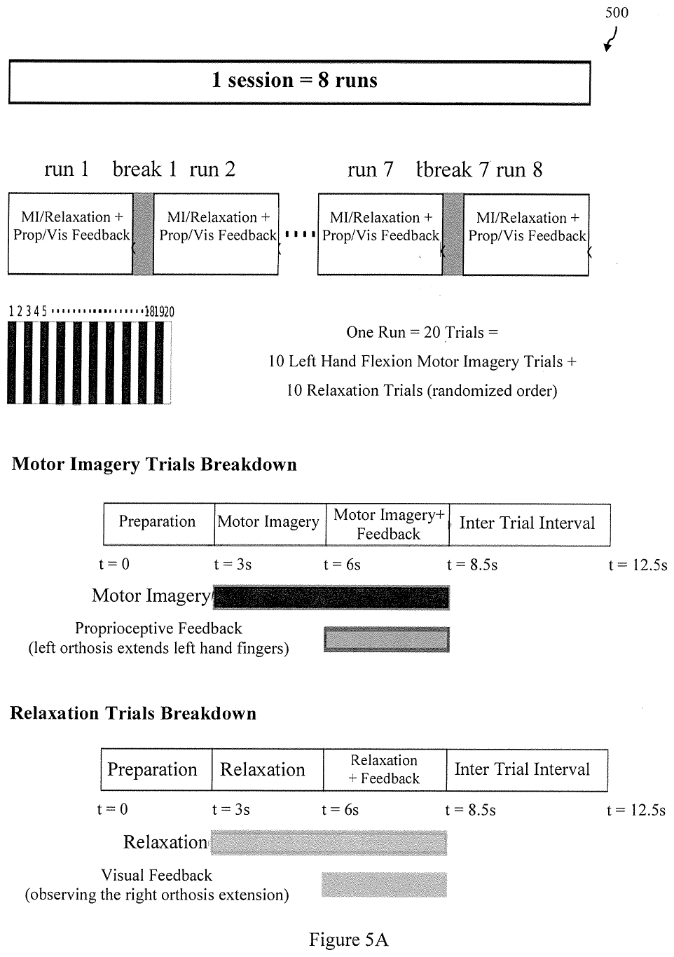

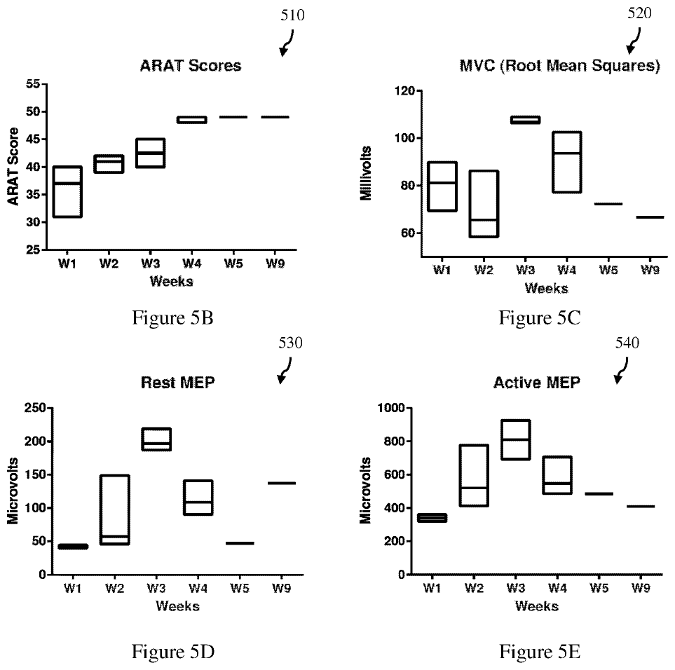

Every session comprised of eight runs of 20 trials (ten left hand finger extension motor imagery, and ten relaxation trials). Following a stroke, the fingers often assume a flexed position and to obtain useful function in the hand, strengthening control of the finger extensors is desirable. Therefore, unlike the last phases of our studies with healthy populations, here we rewarded the motor imagery of the stroke patient by extending his fingers. FIG. 5A is a schematic plot of a trial of a BCI rehabilitation method according to an embodiment demonstrates the time course of neurofeedback training sessions.

Each trial started with an auditory cue at t=0 s, followed by another auditory command at t=3 s, which instructed the patient to perform relaxation or MI of left-hand finger extension. After 3 s of MI/relaxation performance, feedback provision started and was updated every 16/24/48/96 ms according to the randomized and predetermined FUI value for each session (see below). At t=8.5 s, the trial finished and after a 4 s inter-trial interval, the next trial started.

A 16th-order autoregressive model was built according to the spectral power of the most recent 500 ms time window of EEG signals. The coefficients of the autoregressive models were then used to classify whether an event-related (de)synchronisation (ERD/ERS) at 15 Hz (the optimal frequency for the subject) occurred. At each FUI, if an ERD was detected, the left orthosis extended the left-hand four fingers to a fraction of a degree and rewarded MI with proprioceptive feedback. For relaxation trials, however, it was an ERS that extended the right orthosis to a fraction of a degree and rewarded relaxation trials with visual feedback through observation of the right orthosis extension.

Functional changes were monitored using the Action Research Arm Test (ARAT). We also used rest and active MEP as well as MVC as our secondary neurophysiological tests. FIG. 5B is a plot of Action Research Arm Test (ARAT) scores for a stroke patient over the course of the trial 510 illustrated in FIG. 5A according to an embodiment. FIG. 5C is a plot of Maximum Voluntary Contraction (MVC) calculated using root mean squares of EMG signals for a stroke patient over the course of the trial 520 illustrated in FIG. 5A according to an embodiment. FIG. 5D is a plot of Resting Motor Evoked Potential (MEP) scores for a stroke patient over the course of the trial 530 illustrated in FIG. 5A according to an embodiment. FIG. 5E is a plot of Active Motor Evoked Potential (MEP) scores for a stroke patient over the course of the trial 540 illustrated in FIG. 5A according to an embodiment.

In the first week of BCI training (week 2) the average ARAT score was 40.75 that compared to its baseline value in week 1 (36), showed a 13% increase. The ARAT scores reached 42.5 in week 3 (no BCI training) and showed an 18% increase compared to the reference ARAT score of 36. The average ARAT score in week 4, in which another round of BCI training took place, was 48 and revealed a 34% increase compared to the baseline value. In week 5, the ARAT score revealed a subtle increase of less than 2% and reached to 49. The increasing trend of the ARAT scores changed after week 5, and in week 9 its value plateaued at 49. Overall, the ARAT scores revealed a 36% increase over weeks 1-5 and then plateaued at week 9 where the highest increments occurred in weeks with BCI training (week 2 and week 4). FIG. 5B demonstrates the weekly averages and standard deviations of the ARAT scores.

Maximum voluntary contraction (MVC), that was measured by calculating the root mean squares of the EMG signals, was found to be 80 mV in the week 1 (considered as reference value). In week 2, however, The MVC decreased by 16% and was measured at 68 mV. In week 3 the decreasing trend of the MVC changed and with a 34% increase compared to its reference value reached to 108 mV. In week 4, even though it dropped to 91 mV and its value became smaller than that of week 3, it still remained 14% higher than the baseline. In week 5 and week 9, it went again below baseline and reached to 72 mV and 67 mV, respectively. Altogether, the MVC scores were only above the baseline value in weeks 3-4. FIG. 5C summarizes the MVC values across weeks 1-9.

Measuring the rest MEP in week 1, it showed to be 43 μV. In week 2 it almost doubled (increased by 96%) and reached to 84 μV. Then, in week 3 it revealed the highest rise with 271% increase and became 203 μV. In week 4, even though it was still 164% larger than its reference value in week 1, it dropped to 113 μV. The decremental trend continued in week 5 where the rest MEP was measured at 47 μV and was just 9% larger than its baseline value. However, in week 9 its value raised to 137 μV and showed to be 219% larger than its reference value. Overall, the rest MEP had a rising trend over weeks 2-3 followed by a decrement along weeks 4-5, and finally ended up with an increase in week 9. FIG. 5D depicts the rest MEP values measured along the study.

In week 1 the baseline peak to peak value of the active MEP, where the participant was applying 150 grams of force, was 340 μV. In week 2, the active MEP showed a 67% increase and reached to 570 μV. In week 3 the active MEP value raised for the second time and reached to 809 μV and showed a 138% increase compared with its baseline value. In week 4, the active MEP dropped to 580 μV, but it was still 70% larger than the reference. The decrement continued in week 5 and week 9 where the active MEPs were measured at 484 and 408 μV, though they were still above the baseline level by 42 and 20%, respectively. To sum up, active MEPs, had an increasing trend along weeks 2-3, followed by a decreasing trend across weeks 4-9. FIG. 5E shows the active MEPs trend over the study course.

The main finding of this case study is that neurofeedback training with FUIs chosen within 16-96 ms range may potentially have a constructive impact on the motor behaviour following stroke. The mentioned possibility is supported by the reported 36% increase in the ARAT scores that was achieved after 10 sessions of neurofeedback training It is noted that any increase in ARAT scores of more than 10% is considered clinically significant. Other studies on application of restorative BCIs for stroke rehab with real time proprioceptive feedback adopted larger values for the FUI (eg 200 ms or 300 ms). However, FUI values in the current study (16, 24, 48 , or 96 ms were at least two times faster than previous studies and provided improved results. It is hypothesised that the adopted shorter FUIs may have enhanced neuroplasticity that was manifested in the observed increment in the ARAT scores.

As described herein, embodiments of BCI rehabilitation methods focus on the use of shorter than usual Feedback Update Intervals (FUI) for motor imagery based treatment. Optimisation of FUI has not been extensively studied and prior studies on the use of BCI for stroke rehabilitation using real-time proprioceptive feedback have typically used FUIs in the 200-300 ms range. In contrast embodiments of the present system use much shorter FUI values of less than 100 ms, and this has been shown to produce a clinically significant improvement over the course of treatment. Whilst not being bound by theory, it is believed that the use of short FUI values facilitate Hebbian learning (ie neuroplasticity), as proprioceptive signals (ie induced movement feedback signals) are provided to the brain at a sufficient rate to trigger long term potentiation and strengthening of synaptic efficacy between co-activated motor neurons leading (ie neuroplasticity) to improved patient outcomes.

Further embodiments also measure reaction times of patients and select the minimum FUI value based on the measured reaction time of the unaffected limb with (faster) reaction times indicating the use of shorter FUI values. Again, whilst not being bound by theory, it is considered that reaction times indicate the underlying ability of the patient to respond to feedback, and for patients with comparatively slower reaction times, they are initially unable to respond to fast feedback. Thus by measuring the patients' reaction time, the FUI can be tuned to an optimal value for the patient. Further this can be repeated and the FUI adjusted over the course of a treatment (ie measured between sessions and used to adjust the FUI for a session). A further embodiment measures the reaction time of the affected limb of the patient and used to set the initial reaction time. For many patients with significant impairment, the course of treatment begins with conventional long FUIs in the range of 100 ms to 1000 ms with the maximum determined based on the measured reaction time. For patients with no residual motor function the FUI is set to a maximum initial value. The FUI is progressively shortened over the course of treatment until the FUI is dropped below 100 ms. For patients with severe impairment, they are initially unable to respond to fast (sub 100 ms) FUI intervals and thus this approach allows operant learning in the earlier stages of treatment. In this initial stage they hear or see the instruction and the body part is moved. This restores basic motor control, and then as the patient improves (and reaction times drop), the patient can be transitioned to FUI values of under 100 ms where longer term Hebbian learning can occur. Changes in the FUI can be based on various measures of improvement, such as based on accuracy, ERD, reaction time, or some combination. The method may be used for limbs or part of limbs, including individual fingers. In these embodiments the patient is asked to perform motor imagery of a specific finger or a number of fingers (target fingers) and gets rewarded by extension of that finger only (individual finger training), or all target fingers. Embodiments of the method and system may be used in any post-stroke phases including acute, sub-acute, and chronic phases. The method may also be used for patients with symptoms or diseases similar to stroke.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

Those of skill in the art would understand that information and signals may be represented using any of a variety of technologies and techniques. For example, data, instructions, commands, information, signals, bits, symbols, and chips may be referenced throughout the above description may be represented by voltages, currents, electromagnetic waves, magnetic fields or particles, optical fields or particles, or any combination thereof.

The processing of signals may be performed directly in hardware, in a software module executed by a processor, or in a combination of the two. For a hardware implementation, processing may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described herein, or a combination thereof. Software modules, also known as computer programs, computer codes, or instructions, may contain a number of source code or object code segments or instructions, and may reside in any computer readable medium such as a RAM memory, flash memory, ROM memory, EPROM memory, registers, or any suitable form of computer readable medium.

In one embodiment the processing is performed by a computer apparatus 30 comprising one or more central processing units (CPU), a memory, and an Input/Output (or Communications) interface, and may include a graphical processing unit (GPU), and input and output devices. The CPU may comprise an Input/Output Interface, an Arithmetic and Logic Unit (ALU) and a Control Unit and Program Counter element. The Input/Output Interface may comprise lines or inputs for receiving signals or data from the load cell module, switch module, indicator module and communications module. The communications interface is configured to communicate with a communications module in another device using a predefined communications protocol which may be wireless or wired (eg Bluetooth, Zigbee, IEEE 802.15, IEEE 802.11, TCP/IP, UDP, etc).

The computing apparatus 30 may comprise a single CPU (core) or multiple CPU's (multiple core), or multiple processors. The computing apparatus may be a server, desktop or portable computer and may use a parallel processor, a vector processor, or may be part of a distributed (cloud) computing apparatus. The memory 34 is operatively coupled to the processor(s) 32 and may comprise RAM and ROM components, and secondary storage components such as solid state disks and hard disks, which may be provided within or external to the device. The memory may comprise instructions to cause the processor to execute a method described herein. The memory may be used to store the operating system and additional software modules or instructions. The processor(s) may be configured to load and execute the software code, modules or instructions stored in the memory. The computing apparatus may comprise additional electronic modules or boards to perform signal conditioning or pre-processing, sample and hold, and signal processing. The computing apparatus may include a chargeable battery.

As used herein, a phrase referring to “at least one of” a list of items refers to any combination of those items, including single members. As an example, “at least one of: a, b, or c” is intended to cover: a, b, c, a-b, a-c, b-c, and a-b-c.

The reference to any prior art in this specification is not, and should not be taken as, an acknowledgement of any form of suggestion that such prior art forms part of the common general knowledge.

It will be appreciated by those skilled in the art that the disclosure is not restricted in its use to the particular application or applications described. Neither is the present disclosure restricted in its preferred embodiment with regard to the particular elements and/or features described or depicted herein. It will be appreciated that the disclosure is not limited to the embodiment or embodiments disclosed, but is capable of numerous rearrangements, modifications and substitutions without departing from the scope as set forth and defined by the following claims.

Please note that the following claims are provisional claims only, and are provided as examples of possible claims and are not intended to limit the scope of what may be claimed in any future patent applications based on the present application. Integers may be added to or omitted from the example claims at a later date so as to further define or re-define the scope.

1. A Motor Imagery (MI) based Brain-Computer Interface (BCI) rehabilitation method, the method comprising:
performing a plurality of trials wherein the plurality of trials are broken into a plurality of sessions, each session comprising a plurality of trial runs, and each trial run comprises a set of consecutive trials using the same Feedback Update Interval (FUI), and each trial comprises:
providing an auditory or visual stimulus to a user to instruct them to imagine performing a physical action with a body part during a trial period, wherein the body part is an affected limb or part of an affected limb;
periodically processing one or more signals from one or more sensors configured to record the electrical or magnetic activity of the brain or the brain metabolism during the trial period at the FUI, and processing the one or more signals is performed in a time less than the FUI and comprises:
determining if a Motor Imagery (MI, intention to perform the instructed action) was formed during a sampling window;
generating a BCI output signal to actuate an output apparatus to move the user's body part if it is determined that a MI was formed to provide proprioceptive feedback to the user;
measuring one or more reaction times of a user and determining the FUI for one or more trials is based on one or more measured reaction times; and
obtaining a measure of improvement after one or more trial runs, and using the measurement of improvement to adjust the FUI for a subsequent plurality of trial runs.

2. The method as claimed in claim 30, wherein measuring one or more reaction times comprises measuring a reaction time of the corresponding unaffected limb or part of an unaffected limb of the user prior to performing one or more trials, and wherein determining the FUI interval for the one or more trials based upon the measured reaction time is performed such that reaction times are positively correlated with FUI values such that shorter reaction times generate shorter FUIs.

3. (canceled)

4. The method as claimed in claim 2, wherein the FUI is reduced for a subsequent plurality of trial runs until the FUI value reaches a lower limit, where the lower limit is determined from the measured reaction time of the corresponding unaffected limb or part of the unaffected limb.

5. The method as claimed in claim 1, wherein the step of measuring one or more reaction times comprises measuring a reaction time of the affected limb or part of the affected limb of the user if there is residual motor function in the affected limb or part of the affected limb and if the measured reaction time is greater than a first threshold, then the FUI interval is set to an initial FUI value between 100 ms and an upper value, and if there is no residual motor function in the affected limb or part of the affected limb, the FUI value is set to the upper value.

6. The method as claimed in claim 1, wherein the consecutive trials within a run, now labelled motor imagery trials are interspersed with relaxation trials during which the user does not imagine moving the body part, and Event Related Desynchronisation (ERD) times are calculated based on the difference between the spectral power of the motor imagery trials and relaxation trials within a trial run and one of the measures of improvement is based on the ERD during the trial run.

7. (canceled)

8. The method of claim 1, wherein the measurement of improvement is based on one or more of an accuracy measure based upon the number of trials where the user exceeds a threshold level of movement of the body part, measuring one or more reaction times of a user, or taking a plurality of measurements of an active motor evoked potential (MEP) of the user.

9-14. (canceled)

15. The method as claimed in claim 1 wherein determining if the motor imagery of the instructed action was formed during a sampling window comprises detecting event related desynchronization (ERD) in the sensorimotor cortex using the one or more BCI input signals from one or more sensors, and wherein the one or more signals from one or more sensors are electroencephalography (EEG) signals from a plurality of EEG sensor electrodes placed on the skull of the user.

16. (canceled)

17. The method as claimed in claim 15, wherein determining if the Motor Imagery (MI, intention to perform the instructed action) was formed during a sampling window comprises pre-processing the one or more signals to reduce noise and/or artefacts, performing feature extraction on the pre-processed one or more signals, post-processing extracted features to improve feature distribution and/or mitigate redundancy, and using a feature translator to determine if the extracted features indicate the Motor Imagery (MI, intention to perform the instructed action) was formed during the sampling window.

18-20. (canceled)

21. The method as claimed in claim 1 wherein the BCI output signal is a binary signal, and the output apparatus incrementally moves the body part if a first signal is received, and the output apparatus does not move the body part if a second signal is received.

22. (canceled)

23. A Motor Imagery (MI) based Brain-Computer Interface (BCI) rehabilitation system comprising:
one or more sensors configured to record the electrical or magnetic activity of the brain or the brain metabolism during the trial period and generate one or more BCI input signals;
a computing apparatus comprising an output indicator device, a processor and a memory; and
an output apparatus in communication with the computing apparatus and comprising a body part support and a motor configured to incrementally move the body part support between two positions in response to one or more BCI output signals received from the computing apparatus,
wherein the BCI input signals are provided as input to the computing apparatus, and the memory comprises instructions to configure the processor to perform a plurality of BCI trials, and wherein the plurality of trials are broken into a plurality of sessions, each session comprising a plurality of trial runs, and each trial run comprises a set of consecutive trials using the same Feedback Update Interval (FUI), and each trial comprises:
providing an auditory or visual stimulus to a user using the output indicator device to instruct them to imagine performing a physical action with a body part during a trial period, wherein the body part is an affected limb or part of an affected limb;
periodically processing one or more signals from the one or more sensors configured to record the electrical or magnetic activity of the brain or the brain metabolism during the trial period at the FUI, and processing the one or more signals is performed in a time less than the FUI and comprises:
determining if a Motor Imagery (MI, intention to perform the instruction was) was formed during a sampling window;
generating a BCI output signal to actuate the output apparatus to move the user's body part if it is determined that a MI was formed to provide proprioceptive feedback to the user;
measuring one or more reaction times of a user and determining the FUI for one or more trials is based on one or more measured reaction times; and
obtaining a measure of improvement after one or more trial runs, and using the measure of improvement to adjust the FUI for a subsequent plurality of trial runs.

24. The system as claimed in claim 23, wherein the one or more sensors configured to record the electrical or magnetic activity of the brain or the brain metabolism during the trial period comprises:
a wearable apparatus comprising a plurality of electroencephalography (EEG) sensor electrodes;
an amplifier configured to receive and amplify the signals from the plurality of EEG sensor electrodes to generate the one or more BCI input signals.

25. The system as claimed in claim 23, wherein the body part is a hand, and the motor is a servomotor and the body part support is a servo motor controlled flexible orthosis configured to support a hand and move fingers from a fully flexed position to a fully extended position in a series of incremental steps.

26. The system as claimed in claim 23, wherein the output apparatus further comprises a visual feedback component comprising a servomotor controlled orthosis configured to move a flexible member, whilst not engaged with a body part, from a fully flexed position to a fully extended position in a series of incremental steps.

27. The system as claimed in claim 24, further comprising a force sensor, a transcranial magnetic stimulation machine, and an electromyogram amplifier.

28. A computer readable medium comprising instructions for causing a processor to perform a Motor Imagery (MI) based Brain-Computer Interface (BCI) rehabilitation method comprising:
performing a plurality of trials wherein the plurality of trials are broken into a plurality of sessions, each session comprising a plurality of trial runs, and each trial run comprises a set of consecutive trials using the same Feedback Update Interval (FUI), and each trial comprises:
providing an auditory or visual stimulus to a user to instruct them to imagine performing a physical action with a body part during a trial period, wherein the body part is an affected limb or part of an affected limb;
periodically processing one or more signals from one or more sensors configured to record the electrical or magnetic activity of the brain or the brain metabolism during the trial period at the FUI, and processing the one or more signals is performed in a time less than the FUI and comprises:
determining if a Motor Imagery (MI, intention to perform the instructed action) was formed during a sampling window;
generating a BCI output signal to actuate an output apparatus to move the user's body part if it is determined that a MI was formed to provide proprioceptive feedback to the user;
measuring one or more reaction times of a user and determining the FUI for one or more trials is based on one or more measured reaction times; and
obtaining a measure of improvement after one or more trial runs, and using the measure of improvement to adjust the FUI for a subsequent plurality of trial runs.

29. The method as claimed in claim 1, wherein during the plurality of trials, the FUI is reduced to less than 100 ms.

30. The method as claimed in claim 29, wherein using the measure of improvement to adjust the FUI comprises reducing the FUI value if the measure of improvement exceeds a threshold value.

31. The system as claimed in claim 23, wherein during the plurality of trials, the FUI is reduced to less than 100 ms.

32. The system as claimed in claim 31, wherein measuring one or more reaction times comprises measuring a reaction time of the corresponding unaffected limb or part of an limb of the user prior to performing one or more trials, and wherein determining the FUI interval for the one or more trials based upon the measured reaction time is performed such that reaction times are positively correlated with FUI values such that shorter reaction times generate shorter FUIs.

33. The system as claimed in claim 32, the FUI is reduced for a subsequent plurality of trial runs until the FUI value reaches a lower limit, where the lower limit is determined from the measured reaction time of the corresponding unaffected limb or part of a limb.

34. The system as claimed in claim 23, wherein the step of measuring one or more reaction times comprises measuring a reaction time of the affected limb or part of affected limb of the user if there is residual motor function in the affected limb or part of affected limb and if the measured reaction time is greater than a first threshold, then the FUI interval is set to an initial FUI value between 100 ms and an upper value, and if there is no residual motor function in the affected limb or part of affected limb, the FUI value is set to the upper value.

35. The system as claimed in claim 23, wherein the consecutive trials within a run, now labelled motor imagery trials are interspersed with relaxation trials during which the user does not imagine moving the body part, and Event Related Desynchronisation (ERD) times are calculated based on the difference between the spectral power of the motor imagery trials and relaxation trials within a trial run and one of the measures of improvement is based on the ERD during the trial run.

36. The system as claimed in claim 23, wherein the measurement of improvement is based on one or more of an accuracy measure based upon the number of trials where the user exceeds a threshold level of movement of the body part, measuring one or more reaction times of a user, or taking a plurality of measurements of an active motor evoked potential (MEP) of the user.

37. The system as claimed in claim 23, wherein determining if the motor imagery of the instructed action was formed during a sampling window comprises detecting event related desynchronization (ERD) in the sensorimotor cortex using the one or more BCI input signals from the one or more sensors.

38. The system as claimed in claim 23, wherein determining if the Motor Imagery (MI, intention to perform the instructed action) was formed during a sampling window comprises pre-processing the one or more signals to reduce noise and/or artefacts, performing feature extraction on the pre-processed one or more signals, post-processing extracted features to improve feature distribution and/or mitigate redundancy, and using a feature translator to determine if the extracted features indicate the Motor Imagery (MI, intention to perform the instructed action) was formed during the sampling window.
